(19) United States
(12) Patent Application Publication
(10) Pub. No.: US 2025/0229086 A1
(43) Pub. Date: Jul. 17, 2025

(54) TREATMENT OF INFLAMMATORY DISORDERS

(71) Applicant: SetPoint Medical Corporation, Valencia, CA (US)

(72) Inventors: Jacob A. LEVINE, West Hempstead, NY (US); David CHERNOFF, Sausalito, CA (US); Murthy V. SIMHAMBHATLA, Valencia, CA (US)

(21) Appl. No.: 18/730,753

(22) PCT Filed: Jan. 20, 2023
(86) PCT No.: PCT/US2023/061048

§ 371 Date: Jul. 19, 2024

Related U.S. Application Data
(60) Provisional application No. 63/266,970, filed on Jan. 20, 2022.
(60) Provisional application No. 63/407,588, filed on Sep. 16, 2022.

(51) Int. Cl. A61N 1/36

(52) U.S. Cl. CPC A61N 1/3606; A61N 1/36014

(57) ABSTRACT

Methods and apparatuses for treating inflammatory diseases by neurostimulation in patients who have failed to adequately respond or have become intolerant to a drug therapy (such as a TNF inhibitor and/or a JAK inhibitor).

### CROSS REFERENCE TO RELATED APPLICATION

This patent application claims priority to U.S. provisional patent application No. 63/266,970, titled “POTENTIATION OF INFLAMMATION INHIBITORY AND IMMUNOSUPPRESSANT DRUGS BY NERVE ACTIVATION,” and filed on Jan. 20, 2022, and to U.S. provisional patent application 63/407,588, titled “TREATMENT OF INFLAMMATORY DISORDERS,” and filed on Sep. 16, 2022, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Rheumatoid arthritis (RA) is a common chronic inflammatory disease that can lead to substantial loss of physical function due to pain and joint destruction. The underlying mechanisms of the disease involves the body's immune system attacking the joints. Although there is currently no cure for RA, treatments can improve symptoms and slow the progression of the disease. Generally, the goals of treatment are to minimize symptoms such as pain and swelling, to prevent bone and joint destruction, in order to maintain day-to-day functioning and reduce the risk of long term disability. Pain medications, steroids, and non-steroidal anti-inflammatory drugs (NSAIDs) are often used to help relieve symptoms. Disease-modifying antirheumatic drugs (DMARDs) include a wide category of drugs that are used not only to reduce signs and symptoms but also to slow down the progression of RA by reducing and inflammation caused by autoimmune attacks. DMARDs include synthetic drugs and biological drugs. Biological drugs are generally those that are manufactured in, extracted from, or semi-synthesized from biological sources. Synthetic DMARDs can be categorized as conventional (csDMARDs) or targeted (tsDMARDs). csDMARDs are drugs that have conventionally been used in the treatment of RA and other inflammatory disorders, while the more recently developed tsDMARDs are targeted to interfere with specific intracellular signaling pathways. Examples of DMARDs include methotrexate, sulfasalazine, leflunomide, hydroxychloroquin, TNF inhibitors (e.g., certolizumab, adalimumab, infliximab and etanercept), abatacept, anakinra, tociluzumab Janus kinase (JAK) inhibitors, and others.

Although medications may help in managing RA and other autoimmune disorders, there are significant drawbacks associated with their chronic use. For example, DMARDs may weaken the body's ability to fight infections, leaving patients at risk of developing serious infections. Medications can also have an associated toxicity depending on the dosage and specific drug reactions by certain patients. Therefore, patients require close monitoring for any suspected toxicity effects, for example, to the kidneys or liver or bone marrow. Further, the effectiveness of some medications may wane over time, which may require administration of higher dosages to reach an effective dose and thereby increasing the risk of serious side effects. In addition, some patients may find certain medications make them feel ill such that they cannot tolerate them. What is needed, therefore, are improved methods for treating RA and other inflammatory and immune disorders compared to pharmaceutical drugs alone.

### SUMMARY OF THE DISCLOSURE

The disclosure relates generally to apparatuses (e.g., systems and devices) and methods of nerve stimulation to enhance effectiveness or replace drugs used to inhibit inflammation in the treatment of inflammatory and immune disorders, such as rheumatoid arthritis (RA).

The present invention relates to methods and apparatuses (systems and devices) for treating inflammatory diseases by neurostimulation even in cases where neurostimulation is contraindicated. Surprisingly, the inventors have found that patients who have failed to adequately respond or have become intolerant to a drug therapy (such as a TNF inhibitor) may robustly respond to a treatment regimen (e.g., releasing neurotransmitters that alter the phenotype of immune cells to decrease TNF) to activate a neuroimmune anti-inflammatory pathway. This is surprising because it is believed that treatments that activate the neuroimmune anti-inflammatory pathway rely on the same biological pathway (or pathways) as drug therapy, and therefore would not work in patients for whom the drug is ineffective or has lost effectiveness. Even more surprising, the inventors have found that the treatment regimen described herein may restore the effectiveness of these same drugs in the patient. Described herein are methods and apparatuses (including apparatuses for performing these methods) for reducing inflammation in a patient that is intolerant to or has failed to adequately respond to a particular drug therapy (e.g., TNF inhibition).

For example, described herein are methods of reducing inflammation in a patient that is intolerant to or has failed to adequately respond to TNF inhibition, the method comprising: administering a treatment regimen to activate a neuroimmune anti-inflammatory pathway in the patient that has failed to adequately respond or is intolerant to a TNF inhibitor therapy. For example, a method of reducing inflammation in a patient that is intolerant to or has failed to adequately respond to TNF inhibition may include: identifying that the patient has failed to adequately respond to TNF inhibitor therapy comprising a drug that inhibits TNF; and administering a treatment regimen by applying electrical energy to activate a neuroimmune anti-inflammatory pathway in the patient.

Failure to respond to a particular therapy (e.g., TNF inhibitor therapy, JAK therapy, etc.), in some cases referred to equivalently as failure to adequately respond, means that the patient had insufficient clinical response, for example, either failing to detectably respond at all, or failed to meet established clinical benchmarks, as described herein. For example failure to respond may refer to a change in the DAS28-CRP that is less than the minimal clinically important difference (MCID) deemed to have an implication in clinical management.

The neuroimmune anti-inflammatory pathway may comprises at least one nerve or nerve endings and at least one type of immune cell. Activating the neuroimmune anti-inflammatory pathway in the patient may comprise administering the treatment regimen to one or more nerve targets comprises one or more of: a cranial nerve, a peripheral nerve, a spinal nerve, or a nerve ending within an organ. Activating the neuroimmune anti-inflammatory pathway in the patient may include administering the treatment regimen within the patient's brain. In some examples activating the neuroimmune anti-inflammatory pathway in the patient comprises administering the treatment regimen to one or more of a vagus nerve, a trigeminal nerve, a splenic nerve, an auricular nerve, or a sacral nerve. Activating the neuroimmune anti-inflammatory pathway in the patient may comprise administering the treatment regimen to a nerve target comprising a peripheral nerve target.

In general, administering the treatment regimen comprises releasing neurotransmitters that alter the phenotype of immune cells to decrease TNF. Releasing neurotransmitters may comprise releasing neurotransmitters that bind to receptors on the immune cells. The neurotransmitters that bind to receptors on the immune cells may be released directly from a stimulated target or by a second nerve that is activated or deactivated through neural-to-neural connections. The neurotransmitters that bind to receptors on the immune cells may be released from a non-neuronal cell. The neurotransmitters released can be one or more of: acetylcholine or norepinephrine or dopamine. The immune cells may be one or more of: monocytes, macrophages, neutrophils, eosinophils, basophils, mast cells, dendritic cells, natural killer cells, B cells or T cells. The neurotransmitters released may bind to one or more of: muscarinic acetylcholine receptors, nicotinic acetylcholine receptors, alpha adrenergic receptors, beta adrenergic receptors, or dopaminergic receptors. The neurotransmitter receptors are one or more of: muscarinic acetylcholine receptors, nicotinic acetylcholine receptors, alpha adrenergic receptors, beta adrenergic receptors, or dopaminergic receptors. In some examples the neurotransmitter receptors are one or more of: M1 acetylcholine receptors, alpha-4 beta-2 nicotinic acetylcholine receptors, alpha-7 nicotinic acetylcholine receptors, alpha adrenergic receptors, beta 2 adrenergic receptors, or dopaminergic receptors.

In particular, administering the treatment regimen may include applying energy to one or more nerve targets. For example, applying energy may include applying one or more of: electrical energy, mechanical energy, and thermal energy. In some examples applying energy comprises applying energy directly through contact or proximity, transdermally applying energy, and/or percutaneously applying energy. In general, applying energy may comprise applying energy with energy steering.

The peripheral nerve target may comprise one or more of: a radial nerve, a median nerve, an ulnar nerve, a femoral nerve, a sciatic nerve, a tibial nerve, a splanchnic nerve, a phrenic nerve, a hepatic nerve, a renal nerve, a splenic nerve, a nerve of the external ear, a greater auricular nerve, a lesser occipital nerve, an auriculotemporal nerve, and an auricular branch of the vagus nerve. The spinal nerve targets may comprise one or more of: a sacral nerve, a cervical nerve, a thoracic nerve and a lumbar nerve.

As mentioned above, activating the neuroimmune anti-inflammatory pathway in the patient may include administering the treatment regimen to one or more nerve targets including nerve ending within an organ. The nerve ending within the organ may be a nerve ending of one or more of: the spleen, the liver, the lymph nodes, the stomach, the small bowel, the large intestine, the pancreas, and the thymus. For example, administering the treatment regimen within the patient's brain may comprise administering the treatment regimen to one or more of: the locus coeruleus, motor nucleus of the vagus nerve, nucleus ambiguus, nucleus basalis, hypothalamus, insula, and basal forebrain.

A peripheral nerve target may comprise one or more of: a radial nerve, a median nerve, an ulnar nerve, a femoral nerve, a sciatic nerve, a tibial nerve, a splanchnic nerve, a phrenic nerve, a hepatic nerve, a renal nerve, a splenic nerve, a nerve of the external ear, a greater auricular nerve, a lesser occipital nerve, an auriculotemporal nerve, or an auricular branch of the vagus nerve. The spinal nerve target may comprise: a sacral nerve, a cervical nerve, a thoracic nerve, a lumbar nerve, or a dorsal root ganglion thereof. The nerve ending within the organ may comprise a nerve ending within one or more of: a spleen, a liver, a lymph node, a stomach, an intestine, a pancreas, or a thymus.

In any of these method administering the treatment regimen may comprise administering a drug that directly or indirectly activates acetylcholine receptors. For example, the drug may comprise an α7nicotinic agonist including (nicotine) and/or an AChE inhibitor. The TNF inhibitor therapy may be one or more of: etanercept, adalimumab, certolizumab pegol, infliximab, golimumab, and biosimilars thereof.

The patient being treated may have an immune or inflammatory disorder (and the methods described herein may be method of treating that disorder). For example, the immune or inflammatory disorder may be ulcerative colitis. The immune or inflammatory disorder may be Crohn's disease. The immune or inflammatory disorder may be one of: rheumatoid arthritis and psoriatic arthritis. The immune or inflammatory disorder may be multiple sclerosis. The immune or inflammatory disorder may be one of: rheumatoid arthritis, bursitis, gouty arthritis, polymyalgia rheumatica, Crohn's disease, ulcerative colitis, multiple sclerosis, coeliac disease, systemic lupus erythematosus, aplastic anemia, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, and vasculitis.

As mentioned, in any of these methods the treatment regimen to activate a neuroimmune anti-inflammatory pathway may be applying electrical energy. For example, applying electrical energy may include applying a plurality of electrical stimulations each having a current no greater than an upper comfort level. The upper comfort level may correspond to a highest current at which the patient is comfortable (e.g., within a range that may be pre-determined to be effective.) Any of these methods may include determining the upper comfort level by: administering test stimulations at progressively increasing currents; and associating the upper comfort level to the current at which the patient first feels discomfort. The methods described herein may include determining a threshold current corresponding to a current at which the patient first experiences perception of stimulus. Applying electrical energy may comprise applying nerve stimulations of less than 50 mA (e.g., 50 mA or less, 45 mA or less, 40 mA or less, 35 mA or less, 30 mA or less, 25 mA or less, 20 mA or less, etc.). In some examples applying electrical energy may comprise applying nerve stimulations of 2 mA or less. In some examples applying electrical energy comprises applying nerve stimulations ranging from 20 μA to 200 μA. Applying electrical energy may comprise applying nerve stimulations ranging from 200 μA and 3 mA. Applying electrical energy may comprise applying a charge ranging from 0.2 nanocoulombs and 5 kilocoulomb (e.g., over 5 minutes or less, over 10 minutes or less, over 15 minute or less, over 20 minutes or less, over 30 minutes or less, over 40 minutes or less, over 45 minutes or less, over 1 hours or less, over 1.5 hours or less, over 2 hours or less, over 2.5 hours or less, over 3 hours or less, over 4 hours or less, etc.).

As mentioned, in any of these methods the treatment regimen to activate a neuroimmune anti-inflammatory pathway may be applying mechanical energy (e.g., vibration (haptic) including ultrasound). Applying mechanical energy may comprise applying focused ultrasound within a frequency range 0.1-20 MHz. Applying mechanical energy may comprise applying focused ultrasound within a power range 0.01-10 mW/mm². Applying mechanical energy may comprise applying focused ultrasound within a pressure range of 10-1000 kPa. Applying electromagnetic energy comprise applying electromagnetic induction within a range of 1-500 A/m.

Applying electrical energy may comprise applying electrical energy from an implanted device. The implanted device may contain a pulse generator and at least one lead that terminates with at least one electrode. The electrode may comprise one or more of: a cuff electrode, a thin film electrode, a temperature-dependent form fitting elastomer, and an in situ setting injected conductive material. The at least one electrode may comprise a linear array of electrodes or a nonlinear array of electrodes. The implanted device may be a leadless device comprising a pulse generator and at least one electrode integrated onto the implanted pulse generator. The implanted device may be a transcutaneously powered leaded devices with a receiver that receives, stores, and/or transforms energy delivered from outside the body. The receiver may receive an electrical transmission, including nearfield, midfield, and far field transmissions, at amplitudes between 0.1 uA-500 mA. In some examples, the receiver may receive focused ultrasound energy at frequencies between 0.1-40 MHz and power between 0.01-50 mW/mm2 and pressure between 10-1000 kPa. In any of these examples, the receiver may receive energy through electromagnetic induction between 0.1-500 A/m.

The transcutaneously powered leaded device may contain at least one lead that terminates with at least one electrode. In some examples the implanted device comprises a leaded device powered by harvested energy with a receiver that receives, stores, and/or transforms energy from within the body.

As mentioned, the administration of the treatment regimen described herein may surprisingly restore activity of the TNF inhibitor drug that had previously become ineffective. Thus, any of these methods may include administering, concurrent with the administration of the treatment regimen to activate the neuroimmune anti-inflammatory pathway, the same TNF inhibitor therapy that the patient had previously failed to adequately respond to.

The methods and apparatuses described herein may be used to increase the efficacy of an existing ongoing drug treatment. In some examples described herein are methods of applying a treatment regime (e.g., stimulation to activate a neuroimmune anti-inflammatory pathway in the patient) where the patient is continuing treatment with a partially effective drug (e.g., a drug having reduced or waning efficacy). Thus, these methods may restore efficacy to a drug, surprisingly including drugs that are believed to operate in the same biological pathways.

For example, a method of reducing inflammation in a patient that is intolerant to or has failed to adequately respond to either TNF inhibition may include: identifying that the patient has failed to adequately respond to TNF inhibitor therapy comprising a drug that inhibits TNF; administering a treatment regimen to activate a neuroimmune anti-inflammatory pathway in the patient by applying electrical energy to one of: a vagus nerve, a splenic nerve, a radial nerve, a median nerve, an ulnar nerve, a femoral nerve, a sciatic nerve, a tibial nerve, a splanchnic nerve, a phrenic nerve, a hepatic nerve, a renal nerve, a greater auricular nerve, a lesser occipital nerve, or an auriculotemporal nerve, wherein applying electrical energy comprises applying charge ranging from 0.2 nanocoulombs and 5 kilocoulomb per hour.

Applying electrical energy may include applying electrical energy directly, transdermally applying electrical energy, or percutaneously applying electrical energy. In some examples applying energy comprises applying energy with energy steering. Applying electrical energy may include applying to one or more of: the vagus nerve, the splenic nerve, the femoral nerve or the sacral nerve. Applying electrical energy may include applying to one or more of the vagus nerve and the splenic nerve, or a nerve that drive activation of the vagus nerve or splenic nerve. In some examples applying electrical energy comprises applying to one or more nerve ending within an organ in one or more of: the spleen, the liver, the lymph nodes, the stomach, the small bowel, the large intestine, the pancreas, and the thymus. The TNF inhibitor therapy may be one or more of: etanercept, adalimumab, certolizumab pegol, infliximab, golimumab, and biosimilars thereof.

These methods (including methods of applying electrical energy to activate a neuroimmune anti-inflammatory pathway, may be used to treat a patient having an immune or inflammatory disorder. The immune or inflammatory disorder may be one of: rheumatoid arthritis, bursitis, gouty arthritis, polymyalgia rheumatica, Crohn's disease, ulcerative colitis, multiple sclerosis, coeliac disease, systemic lupus erythematosus, aplastic anemia, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, and vasculitis.

Applying electrical energy may include applying a plurality of electrical stimulations each having a current no greater than an upper comfort level. The upper comfort level may correspond to a highest current at which the patient is comfortable. Any of these methods may include determining the upper comfort level by: administering test stimulations at progressively increasing currents; and associating the upper comfort level to the current at which the patient first feels discomfort. Applying electrical energy may comprise applying nerve stimulations of 50 mA or less (e.g., 45 mA or less, 40 mA or less, 35 mA or less, 30 mA or less, 25 mA or less, 20 mA or less, etc.). Applying electrical energy may comprise applying nerve stimulations of 2 mA or less. Applying electrical energy may comprise applying nerve stimulations ranging from 20 μA to 200 μA. Applying electrical energy may comprise applying nerve stimulations ranging from 200 μA and 3 mA. Applying electrical energy may comprise applying electrical energy from an implanted device. The implanted device may contain a pulse generator and at least one lead that terminates with at least one electrode. The electrode may comprise one or more of: a cuff electrode, a thin film electrode, a temperature-dependent form fitting elastomer, and an in situ setting injected conductive material. The at least one electrode may comprise a linear array of electrodes or a nonlinear array of electrodes. In some examples the implanted device is a leadless device comprising a pulse generator and at least one electrode integrated onto the implanted pulse generator. The implanted device may be a transcutaneously powered leaded devices with a receiver that receives, stores, and/or transforms energy delivered from outside the body. The receiver may receive electrical transmission, including nearfield, midfield, and far field transmissions at amplitudes between 0.1 uA-500 mA.

Any of these methods may include administering, concurrent with the application of electrical energy, the same drug, or a drug of the same class that inhibits TNF that the patient had previously failed to adequately respond to.

For example, a method of reducing inflammation in a patient that is intolerant to or has failed to adequately respond to either TNF inhibition may include: identifying that the patient has failed to adequately respond to TNF inhibitor therapy comprising a drug that inhibits TNF; administering a treatment regimen to activate a neuroimmune anti-inflammatory pathway in the patient by applying electrical energy to one of: a vagus nerve, a splenic nerve, a radial nerve, a median nerve, an ulnar nerve, a femoral nerve, a sciatic nerve, a tibial nerve, a splanchnic nerve, a phrenic nerve, a hepatic nerve, a renal nerve, a greater auricular nerve, a lesser occipital nerve, or an auriculotemporal nerve; and administering, concurrent with the application of electrical energy, the same drug, or a drug of the same class that inhibits TNF that the patient had previously failed to adequately respond to.

Although the methods described above are specific to patient's that have failed to adequately respond to TNF inhibitor therapy comprising a drug that inhibits TNF, also described herein are methods of treating patients that are intolerant to or has failed to adequately respond to Janus Kinase (JAK) inhibition by activating a neuroimmune anti-inflammatory pathway in the patient that has failed to adequately respond or is intolerant to a JAK inhibitor therapy. This is also surprising, as it is currently believed that drugs to treat inflammation by JAK inhibition are using the same or overlapping pathway as activation of the neuroinflammatory pathway. For example, described herein are methods of reducing inflammation in a patient that is intolerant to or has failed to adequately respond to Janus Kinase (JAK) inhibition, the method comprising: administering a treatment regimen to activate a neuroimmune anti-inflammatory pathway in the patient that has failed to adequately respond or is intolerant to a JAK inhibitor therapy. For example, a method of reducing inflammation in a patient that is intolerant to or has failed to adequately respond to Janus Kinase (JAK) inhibition, may include: identifying that the patient has failed to adequately respond to a JAK inhibitor therapy comprising a drug that inhibits JAK; and administering a treatment regimen by applying electrical energy to activate a neuroimmune anti-inflammatory pathway in the patient.

The neuroimmune anti-inflammatory pathway may include at least one nerve or nerve endings and at least one type of immune cell. Administering the treatment regimen may comprise applying energy to one or more nerve targets. For example, applying energy may comprise applying one or more of: electrical energy, mechanical energy, and thermal energy. Applying energy may include applying energy directly through contact or proximity, transdermally applying energy, or percutaneously applying energy. In any of these examples, applying energy comprises applying energy with energy steering.

In any of these methods, activating the neuroimmune anti-inflammatory pathway in the patient may include administering the treatment regimen to one or more nerve targets comprises one or more of: a cranial nerve, a peripheral nerve, a spinal nerve, or a nerve ending within an organ. Activating the neuroimmune anti-inflammatory pathway in the patient may include administering the treatment regimen within the patient's brain. In some examples activating the neuroimmune anti-inflammatory pathway in the patient comprises administering the treatment regimen to one or more of: a vagus nerve, a trigeminal nerve, a glossopharyngeal nerve, an optic nerve, or a facial nerve. Activating the neuroimmune anti-inflammatory pathway in the patient may include administering the treatment regimen to a nerve targets comprising a peripheral nerve target, such as one or more of: a radial nerve, a median nerve, an ulnar nerve, a femoral nerve, a sciatic nerve, a tibial nerve, a splanchnic nerve, a phrenic nerve, a hepatic nerve, a renal nerve, a splenic nerve, a nerve of the external ear, a greater auricular nerve, a lesser occipital nerve, an auriculotemporal nerve, and an auricular branch of the vagus nerve. The spinal nerve targets may include one or more of: a sacral nerve, a cervical nerve, a thoracic nerve and a lumbar nerve. The nerve ending within the organ may be a nerve ending of one or more of: the spleen, the liver, the lymph nodes, the stomach, the small bowel, the large intestine, the pancreas, and the thymus. In any of these examples administering the treatment regimen within the patient's brain may include administering the treatment regimen to one or more of: the locus coeruleus, motor nucleus of the vagus nerve, nucleus ambiguus, nucleus basalis, hypothalamus, and basal forebrain. The peripheral nerve target may include one or more of: a radial nerve, a median nerve, an ulnar nerve, a femoral nerve, a sciatic nerve, a tibial nerve, a splanchnic nerve, a phrenic nerve, a hepatic nerve, a renal nerve, a splenic nerve, a nerve of the external ear, a greater auricular nerve, a lesser occipital nerve, an auriculotemporal nerve, or an auricular branch of the vagus nerve. The spinal nerve target may comprise: a sacral nerve, a cervical nerve, a thoracic nerve, a lumbar nerve, or a dorsal root ganglion thereof. The nerve ending within the organ may comprise a nerve ending within one or more of: a spleen, a liver, a lymph node, a stomach, an intestine, a pancreas, or a thymus.

The JAK inhibitor therapy may be one or more of: baricitinib, tofacitinib, upadacitinib, filgotinib, ruxolitinib, oclacitinib, peficitinib, fedratinib, delgocitinib, abrocitinib, deucravacitinib, and biosimilars thereof.

These methods may be used to treat a patient having an immune or inflammatory disorder. For example, the patient may have ulcerative colitis. In some examples the immune or inflammatory disorder is Crohn's disease. In some examples the immune or inflammatory disorder is one of: rheumatoid arthritis and psoriatic arthritis. The immune or inflammatory disorder may be multiple sclerosis. The immune or inflammatory disorder may be one of: rheumatoid arthritis, bursitis, gouty arthritis, polymyalgia rheumatica, Crohn's disease, ulcerative colitis, multiple sclerosis, coeliac disease, systemic lupus erythematosus, aplastic anemia, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, and vasculitis.

In any of these methods applying electrical energy may include applying a plurality of electrical stimulations each having a current no greater than an upper comfort level. For example, the upper comfort level may correspond to a highest current at which the patient is comfortable. These methods may include determining the upper comfort level by: administering test stimulations at progressively increasing currents; and associating the upper comfort level to the current at which the patient first feels discomfort. Any of these methods may include determining a threshold current corresponding to a current at which the patient first experiences perception of stimulus.

As mentioned above, in any of these methods, applying electrical energy may include applying nerve stimulations of 50 mA or less (e.g., 50 mA or less, 45 mA or less, 40 mA or less, 35 mA or less, 30 mA or less, 25 mA or less, 20 mA or less, etc.). Applying electrical energy may comprise applying nerve stimulations of less than 2 mA. In some examples applying electrical energy comprises applying nerve stimulations ranging from 20 μA to 200 μA. Applying electrical energy may comprise applying nerve stimulations ranging from 200 μA and 3 mA. Applying electrical energy may comprise applying a charge ranging from 0.2 nanocoulombs and 5 kilocoulomb (e.g., over 5 minutes or less, over 10 minutes or less, over 15 minute or less, over 20 minutes or less, over 30 minutes or less, over 40 minutes or less, over 45 minutes or less, over 1 hours or less, over 1.5 hours or less, over 2 hours or less, over 2.5 hours or less, over 3 hours or less, over 4 hours or less, over 1 day or less, etc.).

Applying mechanical energy may include applying focused ultrasound within a frequency range 0.1-20 MHz. Applying mechanical energy may comprise applying focused ultrasound within a power range 0.01-10 mW/mm². Applying mechanical energy may comprise applying focused ultrasound within a pressure range of 10-1000 kPa.

Applying electromagnetic energy may comprises applying electromagnetic induction within a range of 1-500 A/m.

Applying electrical energy may comprise applying electrical energy from an implanted device. The implanted device may contain a pulse generator and at least one lead that terminates with at least one electrode. The electrode may comprise one or more of: a cuff electrode, a thin film electrode, a temperature-dependent form fitting elastomer, and an in situ setting injected conductive material. The at least one electrode may comprise a linear array of electrodes or a nonlinear array of electrodes. The implanted device may be a leadless device comprising a pulse generator and at least one electrode integrated onto the implanted pulse generator. In some examples the implanted device is a transcutaneously powered leaded devices with a receiver that receives, stores, and/or transforms energy delivered from outside the body. The receiver may receive electrical transmission, including nearfield, midfield, and far field transmissions at amplitudes between 0.1 uA-500 mA. The receiver may receive focused ultrasound energy at frequencies between 0.1-40 MHz and power between 0.01-50 mW/mm2 and pressure between 10-1000 kPa. The receiver may receive energy through electromagnetic induction between 0.1-500 A/m.

The transcutaneously powered leaded device may contain at least one lead that terminates with at least one electrode. In some examples the implanted device comprises a leaded device powered by harvested energy with a receiver that receives, stores, and/or transforms energy from within the body.

The JAK inhibitor therapy may inhibit activity of one or more of JAK1, JAK2, JAK3, and TYK2.

Any of these methods may include administering, concurrent with the administration of the treatment regimen to activate the neuroimmune anti-inflammatory pathway, the same JAK inhibitor therapy that the patient had previously failed to adequately respond to.

For example, a method of reducing inflammation in a patient that is intolerant to or has failed to adequately respond to Janus Kinase (JAK) inhibition may include: identifying that the patient has failed to adequately respond to a JAK inhibitor therapy comprising a drug that inhibits JAK; administering a treatment regimen to activate a neuroimmune anti-inflammatory pathway in the patient by applying electrical energy to one of: a vagus nerve, a splenic nerve, a radial nerve, a median nerve, an ulnar nerve, a femoral nerve, a sciatic nerve, a tibial nerve, a splanchnic nerve, a phrenic nerve, a hepatic nerve, a renal nerve, a greater auricular nerve, a lesser occipital nerve, or an auriculotemporal nerve, wherein applying electrical energy comprises applying charge ranging from 0.2 nanocoulombs and 5 kilocoulomb per hour.

The patient may be intolerant to or may have failed to adequately respond to Janus Kinase (JAK) inhibition wherein the JAK inhibitor therapy inhibits activity of one or more of JAK1, JAK2, JAK3, and TYK2. As mentioned above, applying electrical energy may comprise applying electrical energy directly, transdermally applying electrical energy, or percutaneously applying electrical energy. Applying energy may include applying energy with energy steering. Applying electrical energy may include applying to one or more of: the vagus nerve, the splenic nerve, the femoral nerve or the sacral nerve.

Applying electrical energy may include applying to one or more of the vagus nerve and the splenic nerve, or a nerve that drive activation of the vagus nerve or splenic nerve.

In any of these methods, applying electrical energy may include applying to one or more nerve ending within an organ in one or more of: the spleen, the liver, the lymph nodes, the stomach, the small bowel, the large intestine, the pancreas, and the thymus.

The JAK inhibitor therapy may be one or more of: baricitinib, tofacitinib, upadacitinib, filgotinib, ruxolitinib, oclacitinib, peficitinib, fedratinib, delgocitinib, abrocitinib, deucravacitinib. The patient may have an immune or inflammatory disorder. For example, the immune or inflammatory disorder may be one of: rheumatoid arthritis, bursitis, gouty arthritis, polymyalgia rheumatica, Crohn's disease, ulcerative colitis, multiple sclerosis, coeliac disease, systemic lupus erythematosus, aplastic anemia, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, and vasculitis.

Applying electrical energy may comprise applying a plurality of electrical stimulations each having a current no greater than an upper comfort level. The upper comfort level may correspond to a highest current at which the patient is comfortable. Any of these methods may include determining the upper comfort level by: administering test stimulations at progressively increasing currents; and associating the upper comfort level to the current at which the patient first feels discomfort. Applying electrical energy may include applying nerve stimulations of 50 mA or less (e.g., 50 mA or less, 45 mA or less, 40 mA or less, 35 mA or less, 30 mA or less, 25 mA or less, 20 mA or less, etc.). For example, applying electrical energy may include applying nerve stimulations of less than 2 mA. Applying electrical energy may comprise applying nerve stimulations ranging from 20 μA to 200 μA. Applying electrical energy may comprise applying nerve stimulations ranging from 200 μA and 3 mA. Applying electrical energy may comprise applying electrical energy from an implanted device. The implanted device may contain a pulse generator and at least one lead that terminates with at least one electrode. In some examples the electrode comprises one or more of: a cuff electrode, a thin film electrode, a temperature-dependent form fitting elastomer, and an in situ setting injected conductive material. The at least one electrode may comprise a linear array of electrodes or a nonlinear array of electrodes. In any of these methods, the implanted device may be a leadless device comprising a pulse generator and at least one electrode integrated onto the implanted pulse generator. The implanted device may be a transcutaneously powered leaded devices with a receiver that receives, stores, and/or transforms energy delivered from outside the body. The receiver may receive electrical transmission, including nearfield, midfield, and far field transmissions at amplitudes between 0.1 uA-500 mA.

Any of these methods may include administering, concurrent with the application of electrical energy, the same drug, or a drug of the same class that inhibits JAK that the patient had previously failed to adequately respond to.

For example, a method of reducing inflammation in a patient that is intolerant to or has failed to adequately respond to Janus Kinase (JAK) inhibition may include: identifying that the patient has failed to adequately respond to a JAK inhibitor therapy comprising a drug that inhibits JAK; administering a treatment regimen to activate a neuroimmune anti-inflammatory pathway in the patient by applying electrical energy to one of: a vagus nerve, a splenic nerve, a radial nerve, a median nerve, an ulnar nerve, a femoral nerve, a sciatic nerve, a tibial nerve, a splanchnic nerve, a phrenic nerve, a hepatic nerve, a renal nerve, a greater auricular nerve, a lesser occipital nerve, or an auriculotemporal nerve; and administering, concurrent with the application of electrical energy, the same drug, or a drug of the same class that inhibits JAK that the patient had previously failed to adequately respond to.

In any of these methods described above, the neurostimulation can be administered according to a nerve stimulation regimen that includes applying a number of nerve stimulations doses to the patient over a period of time. The nerve stimulation regimen may be administered before, during, or after administration of one or more doses of the drug(s). The methods and apparatuses described herein may be useful in treating inflammation associated with rheumatoid arthritis (RA). In some cases, the methods and apparatuses may also be useful in treating other inflammatory and/or immune conditions such as Crohn's disease, ulcerative colitis, multiple sclerosis, coeliac disease, systemic lupus erythematosus, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, vasculitis.

The nerve stimulation treatments described herein may be combined with administration of one or more drugs used to reduce inflammation. In some examples, the drug(s) include one or more immunosuppressant and/or inflammation inhibitory drugs. In some cases, immunosuppressant drug(s) may include one or more disease-modifying antirheumatic drugs (DMARDs). The DMARD(s) can include synthetic drug(s) and/or a biological drug(s). The DMARD(s) can be conventional (csDMARDs) and/or targeted (tsDMARDs). The drug(s) may include one or more enzyme inhibitors (e.g., Janus kinase (JAK) inhibitors), recombinant proteins, vaccines, blood products, gene therapy drugs, antibodies (e.g., monoclonal antibodies), small molecule drugs, and/or cell therapy drugs. In some cases, the drug(s) may be a glucocorticoid, such as prednisone, dexamethasone, and/or hydrocortisone. In some cases the drug(s) may be hydroxychloroquine and/or methotrexate (MTX).

Other drugs may include drugs for Crohn's, such as one or more of: steroids (e.g., beclomethasone dipropionate, budesonide, hydrocortisone, methylprednisolone, prednisolone, prednisone), anti-inflammatory drugs (e.g., balsalazide, mesalamine, olsalazine, sulfasalazine), immunosuppressants (e.g., azathioprine, cyclosporin, mercaptopurine, methotrexate, mycophenolate mofetil, tacrolimus), antibacterial drugs (e.g., ciprofloxacin, metronidazole, rifaximin, vancomycin), biologics and (e.g., adalimumab, infliximab, ustekinumab, vedolizumab). Other drugs may also include drugs for ulcerative colitis, such as one or more of: 5-aminosalicylic acid (5-ASA), balsalazide, mesalamine, olsalazine, sulfasalazine, corticosteroids, immunosuppressants (e.g., 6-mercaptopurine (6-MP), azathioprine, cyclosporine, tacrolimus), biologics (e.g., adalimumab, plus adalimumab-atto, adalimumab-adbm (biosimilars), certolizumab pegol, golimumab, infliximab, ustekinumab, vedolizumab, etc.), tofacitinib, sphingosine 1-phosphate (SiP) receptor modulators, an ozanimod. Other drugs may also include drugs for multiple sclerosis (MS) such as one or more of: Avonex® (interferon beta-1a), Betaseron® (interferon beta-1b), Copaxone® (glatiramer acetate), Extavia® (interferon beta-1b), Glatiramer Acetate Injection (glatiramer acetate—generic equivalent of Copaxone 20 mg and 40 mg doses), Glatopa® (glatiramer acetate—generic equivalent of Copaxone 20 mg and 40 mg doses), Kesimpta® (ofatumumab), Plegridy® (peginterferon beta-1a), Rebif® (interferon beta-1a), Aubagio® (teriflunomide), Bafiertam™ (monomethyl fumarate), Dimethyl Fumarate (dimethyl fumarate—generic equivalent of Tecfidera), Gilenya® (fingolimod), Mavenclad® (cladribine), Mayzent® (siponimod), Ponvory™ (ponesimod), Tecfidera® (dimethyl fumarate), Vumerity® (diroximel fumarate), Zeposia® (ozanimod), Lemtrada® (alemtuzumab), Novantrone® (mitoxantrone), Ocrevus® (ocrelizumab), Tysabri® (natalizumab).

Other drugs may also include drugs for psoratic arthristus (PsA), such as one or more of: etanercept, secukinumab, ustekinumab, adalimumab, infliximab, ixekizumab, guselkumab, risankizumab, certolizumab, abatacept, golimumab, adalimumab, upadacitinib, tofacitinib, sulfasalazine, leflunomide, methotrexate, azathioprine, dexamethasone, prednisone, cortisone, apremilast, mycophenolate mofetil, triamcinolone, corticotropin, cyclosporine, hydroxyurea, acitretin, auranofin.

Nerve stimulation in combination with administration of a drug may allow for decreased dosing of the drug. For example, a first dosage of a drug administered to the patient may be found to inadequately treat a patient's symptoms. A nerve stimulation treatment regimen may be co-administered with the drug at the first dosage to potentiate the drug and effectively treat the patient's symptoms. In some cases, the co-administration of the nerve stimulation may allow for administration of a second dosage, which is less than the first dosage, to effectively treat the patient's symptoms. The co-administration of nerve stimulation and drug therapy may result in better treatment than nerve stimulation alone or drug therapy alone.

In some cases, the dosage of a drug can be tapered when concurrently implemented with nerve stimulation, thereby improving safety and/or minimizing unwanted effects associated with the drug. For example, when on a nerve stimulation therapy, a drug may be tapered by administering it less frequently and/or at a lower concentration. In some cases, the patient may be weaned off the drug altogether.

In some cases, a nerve stimulation regimen may be implemented to effectively treat a patient's symptoms after it has been determined that drug treatment has been unsuccessful, for example, as described above for TNF inhibitor drugs. For example, a method of reducing inflammation in a patient may include determining that administration of a drug on the patient has failed to achieve or to maintain a low disease activity state in the patient and administering a nerve stimulation treatment regimen to achieve the low disease activity state.

A nerve stimulation prescription can be added to a concurrently taken biological or targeted synthetic therapy, or biological or targeted synthetic therapy can be added to an active nerve stimulation treatment prescription, regardless of prior drug exposure.

In some examples, nerve stimulation therapy can reactivate responsivity to a drug in a patient who previously lacked or lost a clinical response to the drug.

In some examples, nerve stimulation therapy can be implemented as an add-on to a drug therapy to improve or extend the clinical efficacy of the drug therapy.

In some cases, a nerve stimulation regimen may be implemented if a patient is determined not to have undergone treatment using a particular drug, or particular class of drugs. For example, a method of reducing inflammation can include determining that the patient has not been treated with a particular drug and applying a nerve stimulation regimen to achieve a low disease activity state in the patient.

For example, the clinical efficacy of nerve stimulation in patients that are naïve to a JAK inhibitor can be superior to nerve stimulation to those with prior JAK inhibitor exposure.

One aspect of the disclosure is a method of reducing inflammation in a patient, the method including: determining that administration of an immunosuppressant drug on the patient has failed to achieve or to maintain a low disease activity state in the patient; and administering a nerve stimulation treatment regimen to achieve the low disease activity state, as described herein.

In this aspect, the method may further include: determining a first dosage that the immunosuppressant drug was administered to the patient prior to administering the nerve stimulation; and wherein administering the nerve stimulation treatment regimen includes concurrently administrating the nerve stimulation treatment regimen and the immunosuppressant drug, wherein the immunosuppressant drug is administered at a second dosage that is less than the first dosage. In this aspect, concurrently administrating the nerve stimulation treatment regimen and the immunosuppressant drug may include administering one type (or more than one type) of immunosuppressant drug.

The nerve stimulation treatment regimen may include a plurality of electrical stimulations each having a current no greater than an upper comfort level. The upper comfort level may correspond to a highest current at which the patient is comfortable.

The method may further include determining the upper comfort level by: administering test stimulations at progressively increasing currents; and associating the upper comfort level to the current at which the patient first feels discomfort.

The method may further include determining a threshold current corresponding to a current at which the patient first experiences perception of stimulus.

The nerve stimulation treatment regimen may include applying nerve stimulations of less than 10 mA. The nerve stimulation treatment regimen may include applying nerve stimulations of less than 2 mA.

The nerve stimulation treatment regimen may include applying nerve stimulations ranging from 20 μA to 200 μA.

The nerve stimulation treatment regimen may include applying nerve stimulations ranging from 200 μA and 2 mA.

The nerve stimulation treatment regimen may include applying a charge ranging from 0.2 nanocoulombs and 5 kilocoulomb.

In this aspect, the immunosuppressant drug may include a disease-modifying antirheumatic drug (DMARD). The DMARD may include one or more of a biological DMARD and a synthetic DMARD. The DMARD may include one or more of a conventional DMARD and a targeted DMARD. The immunosuppressant drug may include one or more of a Janus kinase (JAK) inhibitor and a phosphodiesterase 4 (PDE4) inhibitor.

The immunosuppressant drug may be a biological drug including one or more of etanercept, adalimumab, tocilizumab, abatacept, certolizumab, infliximab, rituximab, golimumab, and a biosimilar thereof.

The immunosuppressant drug may be a non-biologic drug including one or more of ciclosporin, cyclophosphamide, hydroxychloroquine, leflunomide, methotrexate, mycophenolate, and sulfasalazine. The immunosuppressant drug may be a biological drug. The immunosuppressant drug may be a corticotropin (e.g., ACTHAR™ gel)

The method may further include administering a non-biological drug to the patient during administration of the nerve stimulation treatment regimen.

The non-biologic drug may include a steroid. The steroid may include a glucocorticoid. The non-biologic drug may include a nonsteroidal anti-inflammatory drug (NSAID).

The patient may have an immune or inflammatory disorder. The immune or inflammatory disorder may be an autoimmune disorder. The immune or inflammatory disorder may be one or more of rheumatoid arthritis, bursitis, gouty arthritis, polymyalgia rheumatica, Crohn's disease, ulcerative colitis, multiple sclerosis, coeliac disease, systemic lupus erythematosus, aplastic anemia, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, vasculitis, and myocarditis.

Applying the nerve stimulation may include applying the nerve stimulation to one or more of the vagus nerve, the splenic nerve, and the sacral nerve. Nerve stimulation treatment regimen may be administered using an implanted device. Nerve stimulation treatment regimen may include transdermally applying nerve stimulation.

One aspect of the disclosure includes methods of potentiating an immunosuppressant drug in the treatment of an inflammatory disorder in a patient, the method including: co-administrating an immunosuppressant drug therapy regimen and a nerve stimulation regimen on the patient to achieve a low disease activity state in the patient, wherein the immunosuppressant drug therapy regimen includes administration of one or more doses of the immunosuppressant drug, and wherein the nerve stimulation regimen includes administration of a plurality of electrical nerve stimulations. As discussed above, the co-administered drug may be one in which the patient had previously shown little or no (or attenuated/reduced) clinical effect (e.g., a TNF inhibitor, such as certolizumab, adalimumab, infliximab and etanercept, a JAK inhibitor, etc.).

Any of these nerve stimulation regimen may be administered using an implanted device. The nerve stimulation regimen may include transdermally applying the plurality of electrical nerve stimulations. The inflammatory disorder may be one or more of rheumatoid arthritis, bursitis, gouty arthritis, polymyalgia rheumatica, Crohn's disease, ulcerative colitis, multiple sclerosis, coeliac disease, systemic lupus erythematosus, aplastic anemia, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, vasculitis, and myocarditis.

These methods may further include administering a non-biologic drug to the patient during co-administration of the biological drug and the nerve stimulation regimen (e.g., one or more of ciclosporin, cyclophosphamide, hydroxychloroquine, leflunomide, methotrexate, mycophenolate, and sulfasalazine).

The low disease activity state may be determined by a standard method of quantifying symptoms of the patient. A standard method of quantifying symptoms of the patient may include one or more of the Disease Activity Score-28 for rheumatoid arthritis (DAS28-CRP/ESR) and the Clinical Disease Activity Index (CDAI), SDAI, RAPID-3, HAQ-DI, RADAI. A low disease activity state may be determined by measuring one or more biomarkers.

A method of treating a patient with an immune disorder may including: determining that the patient has failed to achieve a low disease state using administration of a first immunosuppressant drug; and co-administrating a nerve stimulation regimen with an immunosuppressant drug therapy regimen to achieve the low disease activity state in the patient, wherein the nerve stimulation regimen includes administration of a plurality of electrical nerve stimulations. The immune disorder may be one or more of rheumatoid arthritis, Crohn's disease, multiple sclerosis, coeliac disease, systemic lupus erythematosus, aplastic anemia, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, vasculitis.

The neurostimulation methods described herein can be adapted to activate the cholinergic anti-inflammatory pathway (NCAP). The nerve stimulation may be administered by an implanted device or may be administered transdermally. In the case of an implanted device, the device may include one or more electrodes that deliver stimulation directly to one or more nerves. In the case of transdermal administration, a transcutaneous electrical nerve stimulation (TENS) device or a focused ultrasound energy device may be adapted to deliver electrical stimulation to stimulate one or more nerves sufficient to activate the cholinergic anti-inflammatory pathway. In some cases, the device(s) is/are configured to administer stimulation according to a prescribed dosage regimen, as described herein.

In some examples, the methods can be implemented by a microstimulator implanted within the patient proximate to one or more nerves, such as a vagus nerve, the sacral nerve, and/or a splenic nerve. A microstimulator can include a controller adapted to deliver electrical stimulation doses at preset times (e.g., throughout the day or week) or as dictated by a user of the device (e.g., on-demand).

The apparatuses (devices and systems) and methods of using them described herein may incorporate some or all of the features of microstimulators, nerve cuffs (“PODs”), chargers, and programmer/controllers described herein may be similar or identical to those described in International Patent Application Publication No. WO 2011/028763, titled “PRESCRIPTION PAD FOR TREATMENT OF INFLAMMATORY DISORDERS;” U.S. Patent Application Publication No. US-2010-0312320-A1, titled “NERVE CUFF WITH POCKET FOR LEADLESS STIMULATOR;” and U.S. Pat. No. 9,993,651, titled “NEURAL STIMULATION DEVICES AND SYSTEMS FOR TREATMENT OF CHRONIC INFLAMMATION,” each of which is herein incorporated by reference in its entirety.

Any of the methods and apparatuses described herein may involve vagus nerve stimulation (VNS) to reduce of inflammation through activation of the cholinergic anti-inflammatory pathway. Any portion of the vagus nerve may be stimulated. In some examples, particular benefit is found when the sub-diaphragmatic vagus nerve and/or the cervical vagus nerve is stimulated.

Treatment and devices for treating inflammation by vagus nerve stimulation has been described, for example, in U.S. Pat. Nos. 6,838,471, 8,914,114, 9,211,409, 6,610,713, 8,412,338, 8,996,116, 8,612,002, 9,162,064, 8,855,767, 8,886,339, 9,174,041, 8,788,034, 9,211,410, and 9,993,651 and in International Patent Publication Nos. WO2016183353A1 and WO2017127758A1, each of which is herein incorporated herein by reference in its entirety.

These and other aspects and advantage are described herein.

### BRIEF DESCRIPTION OF THE DRAWINGS

FIGS. 1-3 are graphs showing clinical results of subjects treated with neuroimmune pathway modulation through nerve stimulation in the treatment of rheumatoid arthritis (RA) or Crohn's disease symptoms that had previous exposure to and lack of efficacy to a TNF or JAK targeted therapy.

FIG. 1 shows clinical results from a subject with RA who had a lack of efficacy to a TNF-targeted therapy prior to starting nerve stimulation.

FIG. 2 shows clinical results from a subject with Crohn's disease who had a lack of efficacy to a TNF-targeted therapy prior to starting nerve stimulation.

FIG. 3 shows clinical results from a subject with RA who had a lack of efficacy to a JAK-targeted therapy prior to starting nerve stimulation.

FIGS. 4A-4B show a table summarizing patient response data as described herein.

FIGS. 5A-5C show a table summarizing patient response data as described herein.

### DETAILED DESCRIPTION

Described herein are methods and apparatuses (e.g., devices and systems) for nerve stimulation to treat inflammatory and autoimmune conditions, such as, but not limited to, rheumatoid arthritis (RA) or Crohn's disease. In particular, described herein is nerve stimulation to treat an inflammatory disorder in patients that are intolerant to, or have failed to adequately respond to, tumor necrosis factor (TNF or sometimes referred to as TNF-α) inhibition. For example, these patients typically failed to adequately respond (above a clinical threshold) to a TNF inhibitor therapy including none or more of: etanercept, adalimumab, certolizumab pegol, infliximab, golimumab, and biosimilars thereof. Surprisingly, the methods and apparatuses described herein for treating a patient believed to be resistant to TNF inhibition therapy is given a treatment regimen to activate a TNF dependent neuroimmune anti-inflammatory pathway (e.g., by the application of electrical, mechanical and/or thermal energy) may result in a significant therapeutic benefit. Further, these methods and apparatuses may restore previously attenuated responses to TNF inhibition pharmaceutical agents. It has long been presumed that treatment regimens, including, e.g., vagus nerve stimulation, would also be ineffective in these patients, since it is thought that the same biological pathways are involved in both pharmaceutical TNF inhibition as in activation of a neuroimmune anti-inflammatory pathway by the application of energy. Specifically, it has long been believed that a patient who has fails to adequately respond substantially to TNF inhibitors would not be benefit from alternative treatments that also block TNF signaling.

The application of energy (e.g., electrical, mechanical or thermal energy) to activate the neuroimmune anti-inflammatory pathway is believed to include the release of TNF; for example, the application of energy to the vagus nerve, trigeminal nerve, or other nerve of the neuroimmune anti-inflammatory pathway (e.g., the glossopharyngeal nerve, optic nerve, or facial nerve(s)) to reduce inflammation reduces release of TNF from immune cells. It is therefore quite surprising that using energy to activate the neuroimmune anti-inflammatory pathway results in a therapeutic benefit in clinical trial subjects that have previously not been adequately treated by TNF inhibitors.

For example, the application electrical energy to activate the neuroimmune anti-inflammatory pathway by stimulation of the vagus nerve with enough energy to depolarize neurons and cause an action potential activates neuroimmune pathways. These neuroimmune pathways utilize both nerves and immune cells, using neurotransmitters to communicate (Tracey (2002), “The inflammatory reflex,” Nature 420(6917): 853-859), and lead to reduction of TNF in immune cells in organs such as the blood, liver, lung, spleen, kidneys, gut and heart (Borovikova et al. (2000), “Vagus nerve stimulation attenuates the systemic inflammatory response to endotoxin,” Nature 405: 458-462; Tracey (2002); Olofsson et al. (2015), “Single-pulse and unidirectional electrical activation of the cervical vagus nerve reduces tumor necrosis factor in endotoxemia,” Bioelectronic Medicine: 37-42). The neuroimmune anti-inflammatory pathway (which may also be referred to as the inflammatory reflex) includes the cholinergic anti-inflammatory pathway and the intestinal cholinergic anti-inflammatory pathway. The cholinergic anti-inflammatory pathway involves the splenic nerve and the spleen (Olofsson et al. (2012), “Rethinking inflammation: neural circuits in the regulation of immunity,” Immunological Reviews 248(1): 188-204) while the intestinal cholinergic anti-inflammatory pathway is spleen independent (Caravaca et al. (2022), “Vagus Nerve Stimulation Reduces Indomethacin-Induced Small Bowel Inflammation,” Frontiers in Neuroscience 15(1842)).

TNF is an important cytokine in the pathogenesis and pathophysiology of many inflammatory diseases, including rheumatoid arthritis, psoriatic arthritis, Crohn's disease, and ulcerative colitis. Indeed, several therapies targeting TNF to decrease its bioavailability have been demonstrated effective and are currently marketed for the treatment of these and other diseases (Monaco et al. (2015), “Anti-TNF therapy: past, present and future,” Int Immunol 27(1): 55-62). VNS has been demonstrated to reduce TNF in immune cells and serum and therapeutic in rheumatoid arthritis (Koopman et al. (2016); Genovese et al. (2020)).

Thus, a patient who has previously not had an adequate clinical response to TNF inhibitors would not be expected to benefit from other treatments that also block TNF signaling. This is supported by multiple published studies showing that treating a patient that has failed to response to a particular TNF inhibitor (drug) with another TNF inhibitor has a significantly lower expected therapeutic potential across all of the clinical parameters studied (Rendas-Baum et al., (2011), “Evaluating the efficacy of sequential biologic therapies for rheumatoid arthritis patients with an inadequate response to tumor necrosis factor-alpha inhibitors,” Arthritis Research & Therapy 13:R25). Further, it has been shown that treating a patient that has insufficient response to a first anti-TNF drug with a non-anti-TNF drug (another class of drug) results in a significantly better response than treating with a second anti-TNF drug (Emery et al. (2015), “Rituximab versus an alternative TNF inhibitor in patients with rheumatoid arthritis who failed to adequately respond to a single previous TNF inhibitor: SWITCH-RA, a global, observational, comparative effectiveness study,” Ann Rheum Dis 74(6): 979-984; Gottenberg et al. (2016), “Non-TNF-Targeted Biologic vs a Second Anti-TNF Drug to Treat Rheumatoid Arthritis in Patients With Insufficient Response to a First Anti-TNF Drug: A Randomized Clinical Trial,” JAMA 316(11): 1172-1180; Choi et al. (2021) “Association of first, second, and third-line bDMARDs and tsDMARD with drug survival among seropositive rheumatoid arthritis patients: Cohort study in A real world setting,” Semin Arthritis Rheum 51(4): 685-691). It is therefore quite surprising that, as shown herein for the first time, using energy to activate the neuroimmune anti-inflammatory pathway (e.g., including the vagus nerve) has been observed to be therapeutically beneficial in clinical trial subjects that have previously not been adequately treated by TNF inhibitors.

The application of energy to activate the neuroimmune anti-inflammatory pathway includes applying energy to nerves other than the vagus nerve, such as the splenic nerve, sacral nerve, etc. and is herein described as being similarly effective. For example, stimulating the splenic nerve is known to cause the release of norepinephrine which acts on adrenergic receptors on immune cells in the spleen to decrease the production and release of TNF (Kees et al. (2003), “Via beta-adrenoceptors, stimulation of extrasplenic sympathetic nerve fibers inhibits lipopolysaccharide-induced TNF secretion in perfused rat spleen,” J Neuroimmunol 145(1-2): 77-85). In addition, norepinephrine also acts on adrenergic receptors within a subset of T cells and B cells that result in production and release of acetylcholine by those cells (Rosas-Ballina et al (2011), “Acetylcholine-synthesizing T cells relay neural signals in a vagus nerve circuit,” Science 334(6052): 98-101). The acetylcholine then acts through the alpha 7 nicotinic acetylcholine receptor on nerves and other immune cells to decrease the production and release of TNF (Vida et al. (2011), “alpha7-cholinergic receptor mediates vagal induction of splenic norepinephrine,” J Immunol 186(7): 4340-4346). The splenic nerve is activated by vagus nerve stimulation causing reduction in TNF production and release (Olofsson et al. (2015). “Single-pulse and unidirectional electrical activation of the cervical vagus nerve reduces tumor necrosis factor in endotoxemia.” Bioelectronic Medicine: 37-42). Based on the current understanding of the neuroimmune anti-inflammatory pathway, it would be expected that a patient who has previously not had an adequate clinical response to TNF inhibitors would not be very sensitive to alternative treatments that also block TNF signaling such as neuroimmune anti-inflammatory pathway activation. It is therefore quite surprising that, as described herein, using energy to activate the vagus nerve and thereby activating the splenic nerve has been observed to be therapeutically beneficial in clinical trial subjects that have previously not been adequately treated by TNF inhibitors.

Similar results would be expected for applying energy to the neuroimmune anti-inflammatory pathway via the sacral nerve, which is part of the parasympathetic nervous system. It has been demonstrated that stimulating the sacral nerve with energy can lead to activation of the vagus nerve. This leads to release of neurotransmitters that act on immunocytes thereby decreasing the production and release of TNF (Pasricha et al. (2020), “Sacral nerve stimulation prompts vagally-mediated amelioration of rodent colitis,” Physiol Rep 8(1): e14294). One would think that a patient who has previously not had an adequate clinical response to TNF inhibitors would not be very sensitive to alternative treatments that also block TNF signaling. Again, it is therefore surprising that, as described herein, using energy to activate the sacral nerve (and therefore the vagus nerve) would be therapeutically beneficial in subjects that have previously not been adequately treated by TNF-inhibitors.

Similarly, the application of energy to activate the neuroimmune anti-inflammatory pathway via the tibial and peronial nerves that branch off at the ST36 acupoint would also not be expected to be effective in subjects that have previously not been adequately treated by TNF-inhibitors (Liu et al. (2021), “A neuroanatomical basis for electroacupuncture to drive the vagal-adrenal axis,” Nature 598(7882): 641-645). This leads to release of neurotransmitters that act on immunocytes thereby decreasing the production and release of TNF. It would therefore be expected that a patient who has previously not had an adequate clinical response to TNF inhibitors would not respond to alternative treatments that also block TNF signaling. It is therefore quite surprising that, as described herein, using energy applied to these nerves, which is known to also activate the vagus nerve, has been observed to be therapeutically beneficial in clinical trial subjects that have previously stopped responding (or simply did not respond) to treatment by TNF-inhibitors.

Although nerve stimulation for treatment of inflammatory conditions has previously been described, nerve stimulation specifically in patient's that have failed to adequately respond or that are intolerant to a TNF inhibitor therapy has not been suggested. As described herein, and illustrated in the clinical examples below, the application of energy to activate the neuroimmune anti-inflammatory pathway in patient's that have failed to adequately respond or that are intolerant to a TNF inhibitor drug therapy is not only effective but may also re-sensitize these same patients to the same TNF inhibitor drug to which they were previously non-responsive. For example, nerve stimulation used in conjunction with administration of one or more types of drugs (e.g., TNF inhibitors) are shown herein to be effective. Concurrent with or following the application of energy to activate the neuroimmune anti-inflammatory pathway may also reduce the required dosage of the TNF inhibitor and/or the necessary frequency of administration of the drug(s) to achieve a therapeutically beneficial effect. In some cases, the nerve stimulation can replace the administration of the drug(s).

The apparatuses and methods described herein may include applying energy to activate the neuroimmune anti-inflammatory pathway at a level necessary to modulate a subject's neuroimmune reflexes. For example, the nerve stimulation may modulate the cholinergic anti-inflammatory pathway (CAP), for example, by modulating cytokine release. The neurostimulation may be administered through the use of implanted neural stimulation devices (microstimulators) and/or transcutaneous electrical nerve stimulation (TENS) devices. The treatment and management of symptoms manifested from the onset of disease (e.g., inflammatory disease) may be based upon modulating neuroimmune reflexes (e.g., the CAP). The CAP pathway normally maintains precise restraint of the circulating immune cells. As used herein, the CAP is a reflex that utilizes cholinergic nerve signals traveling via nerves between the brain, chemoreceptors, and the reticuloendothelial system (e.g., spleen, liver). Local release of pro-inflammatory cytokines (e.g., tumor necrosis factor or TNF) from resident immune cells is inhibited by the efferent, or indirectly by afferent nerve signals. Modulation of the CAP causes important changes in the function and microenvironment of the spleen, liver and other reticuloendothelial organs. Leukocytes which circulate systemically become “educated” as they traverse the liver and spleen are thereby functionally down regulated by the affected environment of the reticuloendothelial system. This effect can potentially occur even in the absence of an inflammatory condition.

Remote inflammation may then be dampened by down-regulated cytokine levels. Stimulation of one or more nerves (e.g., vagus nerve, sacral nerve, and/or splenic nerve) of the neuroimmune anti-inflammatory pathway with a specific regiment of energy (e.g., electrical pulses, thermal energy, mechanical energy, etc.) regulates production of pro-inflammatory cytokines. In-turn, the down regulation of these cytokines may reduce localized inflammation in joints and other organs of patients with autoimmune and inflammatory disorders.

Any of a number of nerves of the neuroimmune anti-inflammatory pathway may be stimulated to activate the cholinergic anti-inflammatory pathway. In some examples, the vagus nerve, the splenic nerve and/or the sacral nerve is/are stimulated. In the case of the vagus nerve, any of a number of regions of the vagus nerve, including nuclei and/or branches of the vagus nerve, may be stimulated. In some examples, one or more of a pharyngeal nerve branch, superior laryngeal nerve branch, superior cervical nerve branch, inferior cervical nerve branch, recurrent laryngeal nerve branch, thoracic nerve branch, branches to the pulmonary plexus, branches to the esophageal plexus, anterior vagal trunk, posterior vagal trunk, dorsal nucleus, nucleus ambiguous, solitary nucleus, and/or spinal trigeminal nucleus of the vagus nerve is/are stimulated. In some specific examples, the efferent vagus nerve is stimulated. In some specific examples, the cervical vagus nerve is stimulated.

The methods and apparatuses described herein may be effective in reducing inflammatory symptoms associated with any of a number of inflammatory disorders and/or autoimmune disorders, including arthritis (e.g., rheumatoid, bursitis, gouty arthritis, and/or polymyalgia rheumatic), inflammatory bowel disease (e.g., Crohn's disease and/or ulcerative colitis), multiple sclerosis, coeliac disease, systemic lupus erythematosus, aplastic anemia, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, vasculitis, and/or myocarditis.

Activation of the cholinergic anti-inflammatory pathway can involve electrical nerve stimulation, pharmacological nerve stimulation, and/or or physical nerve stimulation. Electrical nerve stimulation involves administering of electrical current to the patient to cause sufficient stimulation of one or more nerves to activate the cholinergic anti-inflammatory pathway. The electrical stimulation may be by an implanted device and/or by a transcutaneous device. Pharmacological nerve stimulation involves administering one or more pharmacological agents to the patient sufficient to cause stimulation of one or more nerves. Mechanical nerve stimulation involves one or more mechanical interventions (e.g., acupuncture, focused ultrasound) to cause stimulation of one or more nerves. In some examples, activation of the cholinergic anti-inflammatory pathway involves a combination of electrical, pharmacological and/or physical activation. The electrical, pharmacological and/or physical interventions maybe performed simultaneously or serially during a treatment regimen.

A nerve stimulation treatment regimen can include administering a number of electrical stimulations over a period of time. In some cases, the intensity (which is associated with the electric current) of stimulations can be controlled by the user. In some cases, the frequency of electrical stimulations can be applied upon demand as controlled by the user. For example, a device (e.g., implanted microstimulator or TENS device) can include a control for a user to apply a dose of electrical stimulation when needed. In some examples, a control can be used to increase or decrease an intensity (e.g., current) of the electrical stimulations. In some cases, the intensity and frequency of the electrical stimulations are predetermined. For example, a device (e.g., implanted microstimulator or TENS device) can be programmed to apply electrical stimulation at a prescribed intensity and/or at a prescribed frequency. In one example, the device can be programmed to apply electrical stimulation at a relatively high intensity and/or frequency during times of the day/week/month that are determined to be associated with high pain, and to apply electrical stimulation at a relatively low intensity and/or frequency during times of the day/week/month that are determined to be associated with low pain.

Electrical nerve stimulation may be applied by one or more electrodes of an implanted device (e.g., implanted neurostimulator). The parameters of electrical stimulation may vary depending on the patient, and type and severity of the condition. In some examples, an electrical stimulus of 50 mA or less (e.g., 40 mA or less, 30 mA or less, 20 mA or less, 15 mA or less, 10 mA or less, 2 mA or less, etc.) is used. In some examples, the electrical stimulus can be between about 0.05 to 5 mA, or 0.05 to 10 mA, or 0.05 to 15 mA, 0.05 to 20 mA, or 0.05 to 25 mA or 0.05 to 50 mA. In some examples, the current applied may be less than between about 20 μA to 200 μA against the nerve, or from 200 μA and 2 mA transvenously. In some examples, the charge applied may between about 0.2 nanocoulombs to 1 kilocoulomb.

Transcutaneous electrical nerve stimulation (TENS) can be provided by an electrical stimulation device having at least one electrode that can be placed on the patient's skin. A control or signal generator can deliver the electrical signal stimulus through the electrode. In some examples, the electrical stimulus can be between about 0.05 to 10 mA, or 0.05 to 15 mA, or 0.05 to 20 mA, or 0.05 to 25 mA, or 0.05 to 50 mA. The pulse width can be between about 100 and 1,000 μS. The pulse frequency can be between about 1 and 50 Hz, and the stimulus duration can be between about 1 second and 24 hours. In some examples, the daily charge delivered at about 0.2 nanocoulombs to 1 kilocoulomb per day.

In some examples, dose intensity may be set to within a therapy window. The therapy window is defined by a lower limit of current necessary to trigger an anti-inflammatory response, and an upper limit of current at which the patient feels uncomfortable. The lower limit is referred to as the Threshold (T), and the highest level at which the subject is still comfortable is referred to as the Upper Comfort Level (UCL). The T and/or the UCL may be determined by administering test stimulations to the patient. The testing can involve applying stimulations at progressively increasing (or decreasing) current. The T current can be associated with the current when the patient first experiences perception of stimulation. The UCL current can be associated with the current applied prior to when the patient first feels discomfort.

The clinical results described herein may be measured according to standard clinical measurement tools and techniques. For example, the DAS28-CRP is modified version of the original Disease Activity Score (DAS) for rheumatoid arthritis assessment, which eliminates some of the variables and replaces the erythrocyte sedimentation rate (ESR) with the C-reactive protein (CRP). The Crohn's Disease Activity Index (CDAI) a tool used to quantify the symptoms of patients with Crohn's Disease. Clinical disease activity metrics used for RA (e.g., DAS28-CRP/ESR or CDAI) may be used to determine whether a subject has high disease activity, moderate disease activity, low disease activity, or is in remission. The clinical studies described herein may be implemented according to a treat-to-target approach protocol where a low disease activity state (or remission) is the target.

### Biological Drugs

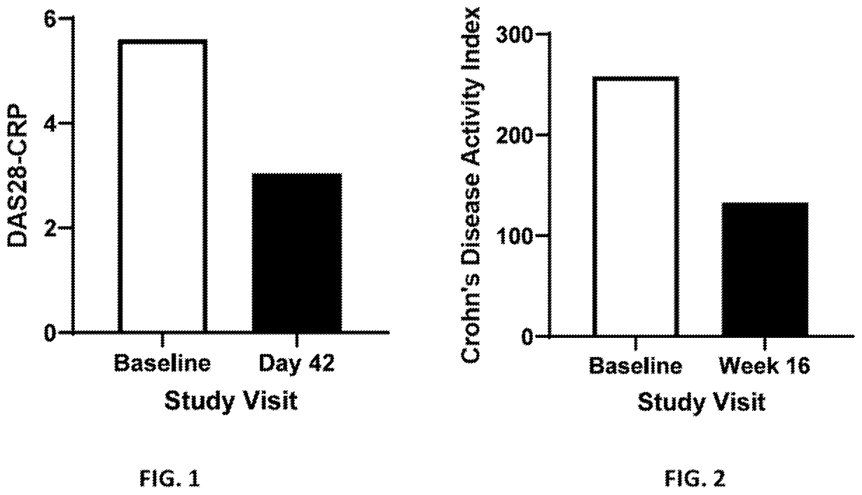

Biopharmaceuticals (also referred to as biologics or biological drugs) generally refer to pharmaceutical drugs manufactured in, extracted from, or semisynthesized from biological sources. Although various biologics have been shown to reduce the signs and symptoms of rheumatoid arthritis, their effectiveness may vary depending on the type of biologic and the patient, and their effectiveness may wane over time. FIGS. 1-2 show clinical results of administration of nerve stimulation therapy without administration of a biologic. In all cases, the subjects were previously found to be insufficiently treated by a biological therapy, specifically a TNF inhibitor. For example, the subjects may have failed to achieve or maintain at least a low disease activity as a target of therapy using the biological therapy alone.

FIG. 1 shows results of clinical studies involving nerve stimulation treatment in RA subjects who had previously been given biologic therapies. The graph of FIG. 1 shows the change in DAS28-CRP from baseline monitored over a period of time (42 day period). The study visit baseline corresponds to the screening visit and study entry. The VNS included electrical stimulation by a microstimulator device implanted in the patient, where patient was given stimulation with simulation intensity was no greater than an Upper Comfort Level (UCL), as described herein. A treatment that results in a change in the DAS28-CRP that is greater than the minimal clinically important difference (MCID) is deemed to have an implication in clinical management. The subject had initially tried and either did not respond or lost response to biological drugs, including anti TNF prior to enrolling into the study Clinical results were recorded as DAS28-CRP scores and associated with “high disease,” “moderate disease,” “low disease,” and “remission” states.

FIG. 1 shows clinical results for a subject with a history of TNF targeted biologic refractory rheumatoid arthritis who enrolled in the VNS study following lack of clinical response to biologics. Specifically, the subject had prior history of being treated and then ceasing treatment of the anti-TNF biologics adalimumab and infliximab and etanercept due to ineffectiveness. (This subject had also not insufficient clinical effectiveness to abatacept.) This subject was a 37 year-old female at baseline, in highly active clinical disease. As shown, the subject had a drop in DAS28-CRP of 2.56 after 42 days of vagus nerve stimulation, representing a clinically meaningful response. These results indicate that therapy with activation of neuro-immune reflexes had efficacy in rheumatoid arthritis despite the subject's previous exposure to and lack of efficacy to a TNF targeted biologic.

FIG. 2 shows clinical results for a subject with a history of TNF targeted biologic refractory Crohn's disease who enrolled in the VNS study following lack of clinical response to biologics. Specifically, the subject had prior history of being treated and then ceasing treatment of the anti-TNF biologics adalimumab and infliximab, golimumab, and etanercept due to ineffectiveness. (This subject had also not insufficient clinical effectiveness to ustekinumab and vedolizumab.) This subject was a 39 year-old female at baseline, in moderately active clinical disease. As shown, the subject achieved clinical remission and had a drop in CDAI of 125 after 16 weeks of vagus nerve stimulation, representing a clinically meaningful response. These results indicate that therapy with activation of neuro-immune reflexes had efficacy in Crohn's disease despite the subject's previous exposure to and lack of efficacy to a TNF targeted biologic.

### Targeted Synthetic Drugs

In general, targeted synthetic DMARDs (tsDMARDs) are drugs that are developed to target a particular molecular structure. Janus kinase (JAK) inhibitors are a class of tsDMARD that target inhibition of one or more of the JAK family of enzymes (JAK1, JAK2, JAK3, TYK2), thereby interfering with the JAK-STAT signaling pathway.

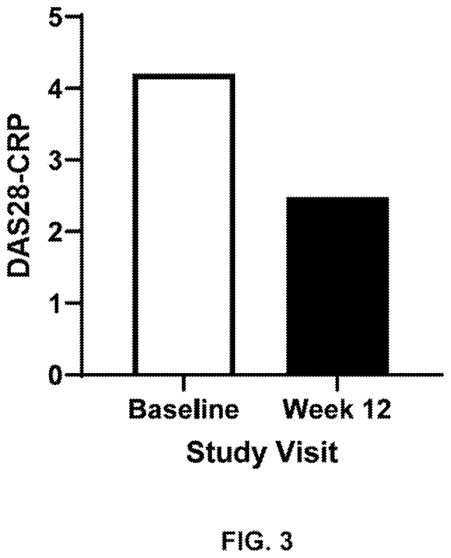

FIG. 3 shows clinical results for a subject with a history of tsDMARD refractory rheumatoid arthritis who enrolled in the VNS study following lack of clinical response to biologics. Specifically, the subject had prior history of being treated and then ceasing treatment of the JAK inhibitor tofacitinib due to ineffectiveness. (This subject had also not insufficient clinical effectiveness to biologics targeting TNF; infliximab, IL-6; tocilizumab, and CTLA-4; abatacept.) This subject was a 47 year-old female at baseline, in highly active clinical disease. As shown, the subject had a drop in DAS28-CRP of 1.72 after 12 weeks of vagus nerve stimulation, representing a clinically meaningful response. These results indicate that therapy with activation of neuro-immune reflexes had efficacy in rheumatoid arthritis despite the subject's previous exposure to and lack of efficacy to a JAK inhibitor.

Although the examples described above involve the use of standard disease activity scores (DAS28-CRP and CDAI) for determining a disease activity state in a subject, other methods may alternatively or additionally be used. For example, a disease state of a subject may alternatively or additionally be quantified by measuring one or more biomarkers. In some cases, the biomarkers may be measured in the blood, urine and/or soft tissue. In some cases, the biomarkers may be patient reported outcomes, quality of life metrics, and/or metrics related to activity or gait. The biomarkers may include proteins, antibodies and/or genetic biomarkers. The type of biomarker being monitored may depend on the particular condition. Example biomarkers may include C-reactive protein, IL6, SAA, rheumatoid factor, autoantibodies against citrullinated proteins erythrocyte sedimentation rate, and/or fecal calprotectin. The biomarker(s) may be measured at various times. For example, the biomarker(s) may be measured before, during and/or after a treatment (e.g., neurostimulation and biologic co-treatment).

Any of the nerve stimulation methods described herein may be combined with one or more immunosuppressant and/or anti-inflammatory drugs. The immunosuppressant drug may include one or more biological drugs (e.g., biological DMARDs), one or more targeted synthetic drugs (e.g., targeted synthetic DMARDs), and/or one or more conventional drugs (e.g., conventional DMARDs). Example biological drugs may include rituximab, tocilizumab, etanercept, abatacept, adalimumab, infliximab, anakinra, certolizumab, certolizumab pegol, golimumab, ixekizumab, natalizumab, secukinumab, ustekinumab, sarilumab, vedolizumab, and/or biosimilars of any of these drugs. Example targeted synthetic drugs may include one or more JAK inhibitors and/or one or more phosphodiesterase 4 (PDE4) inhibitors. Example JAK inhibitors may include upadacitinib, tofacitinib, baricitinib, and/or filgotinib. Example PDE4 inhibitors may include apremilast, roflumilast, apremilast, and/or crisaborole. Example conventional drugs may include ciclosporin, cyclophosphamide, hydroxychloroquine, methotrexate, leflunomide, mycophenolate and/or sulfasalazine. Other possible drugs may include azathioprine, chloroquine, calcineurin inhibitors (e.g., ciclosporin and/or prednisolone), D-penicillamine and/or minocycline. The drugs may include brand-named drugs and/or generic drugs. The drugs may include approved drugs (e.g., by a government agency) and/or pre-approved drugs.

Any of the nerve stimulation and DMARD co-therapies described herein may be administered in combination with one or more additional drugs. Such additional drugs may include one or more corticosteroids (e.g., prednisone, dexamethasone, and/or prednisolone), mTOR inhibitors (e.g., sirolimus, and/or everolimus), IMDH inhibitors (e.g., axathioprine, leflunomide, and/or mycophenolate), monoclonal antibodies (e.g., basiliximab, and/or daclizumab), nonsteroidal anti-inflammatory drugs (NSAID), and/or quinolines (e.g., hydroxychloroquine).

In some examples, the nerve stimulation apparatuses (e.g., microstimulators) may be configured to be inserted or implanted into the body. The apparatus may include a stimulation applicator (also referred to as simply a stimulator) that may be a mechanical and/or electrical stimulator. A mechanical stimulator may be a piezoelectric driver that may vibrate and/or apply pressure to the tissue, including to the nerve, in the nerve treatment parameters, such as mechanical stimulation of the nerve at between 1-2 kHz for a treatment time (e.g., between 1 ms and 5 minutes, e.g., 10 ms-10 sec, etc.). Alternatively or additionally, the stimulation applicator may be an electrical stimulation applicator and may include one or more (e.g., two or more) electrodes configured to apply electrical stimulation to the nerve. For example, electrical stimulation of about 0.1 mA to 10 mA (e.g., between 1 mA-5 mA), at a frequency of between about 1 Hz and about 2 kHz (e.g., between about 1-100 Hz), where the pulses applied have a pulse width of between about (50-500 μsec, e.g., between about 100-300 μsec) or a daily charge delivered at about 0.2 nanocoulombs to 1 kilocoulomb per day. The controller may be configured to enforce an ‘off-time’ following a nerve treatment dose of between about 10 minute and 12 hours (e.g., between about 2 hours and 10 hours, between about 3 hours and 6 hours, at least 2 hours, at least 3 hours, at least 4 hours, at least 5 hours, at least 6 hours, etc.). For example, the stimulator may include an electrode configured to apply electrical energy to the nerve.

In some variations, the apparatus is configured to apply nerve stimulation treatment to the patient in which the nerve stimulation is electrical stimulation. For example, the nerve stimulation treatment may include the application of electrical energy at between about 1-100 Hz (e.g., between about 1-50 Hz, between about 1-20 Hz, between about 5-30 Hz, between about 5-15 Hz, approximately 5 Hz, approximately 10 Hz, approximately 15 Hz, etc.). The energy may have a peak amplitude of between about 0.1 mA and about 2 mA (e.g., between about 0.2 mA and about 1.8 mA, between about 0.5 mA and about 1.5 mA, between about 0.5 mA and about 1 mA, between about 0.1 mA and about 1 mA, approximately 0.5 mA, approximately 0.75 mA, approximately 1 mA, etc.). Alternatively, the applied energy may have an average amplitude of between about 0.1 mA and about 2 mA (e.g., between about 0.2 mA and about 1.8 mA, between about 0.5 mA and about 1.5 mA, between about 0.5 mA and about 1 mA, between about 0.1 mA and about 1 mA, approximately 0.5 mA, approximately 0.75 mA, approximately 1 mA, etc.) or a daily charge delivered at about 0.2 nanocoulombs to 1 kilocoulomb per day. The applied energy is typically pulsed, and may be pulsed square waves, sinusoidal waves, triangular waves, etc. The applied energy may be biphasic or monophasic. For example, the applied energy maybe biphasic. The applied nerve stimulation treatment may be a constant biphasic pulse train having a frequency of between 1-100 Hz (e.g., 10 Hz) and a peak amplitude of between about 0.5 mA and 2 mA (e.g., approximately 0.75 mA). Any of the methods for treatment described herein may be configured to apply this type of nerve stimulation treatment.

Any of the apparatuses (e.g., devices, systems, etc.) described herein may be configured to be implanted on the nerve. Thus, any of these apparatuses may be implanted via a nerve sheath or nerve cuff configured to secure the apparatus onto the nerve and/or prevent movement of the apparatus relative to the nerve and/or insulate the apparatus from other tissues. The implanted apparatus may be implanted in any appropriate location on the nerve. The implant may be a leadless implant that is connected to the nerve (see, e.g., U.S. Pat. Nos. 8,412,338, 8,612,002, 8,886,339, and 8,788,034, each of which is herein incorporated by reference in its entirety). For example, any of these apparatuses may include a nerve cuff configured to secure the stimulator to the nerve. Alternatively, any of these apparatuses may include a lead connecting the microstimulator and/or other components to the stimulation applicator on/around the nerve via one or more leads.

Any of these apparatuses may be configured to apply nerve stimulation treatment including a low duty-cycle electrical stimulation of between about 0.25 mA and about 5 mA to the nerve for less than about 2 minutes. The apparatus may be configured to provide an off-time of at least x minutes/hours (e.g., 10 minutes, 20 minutes, 30 minutes, 40 minutes, 60 minutes, 2 hours, 3 hours, 4 hours, etc.).

Systems for electrically stimulating one or more nerves to treat chronic inflammation may include an implantable, wireless microstimulator and an external charging device. In some examples the system also includes a controller such as a “prescription pad” that helps control and regulate the dose delivered by the system. The microstimulator may be secured in position using a securing device (which may be referred to as a “POD”) to hold the microstimulator in position around or adjacent to a nerve. These microstimulators are designed and adapted for treatment of chronic inflammation and may be configured specifically for such use. Thus, an implantable microstimulator may be small, and adapted for the low duty-cycle stimulation to modulate inflammation. For example, the implantable microstimulator may hold a relatively small amount of power over weeks or even months and discharge it at a rate sufficient to modulate the anti-inflammatory pathway without significantly depressing heart rate or triggering any number of unwanted effects from the nerve.

When configured to sub-diaphragmatic vagus nerve stimulation, the apparatuses may be preferred over cervical vagus nerve application. In such examples, a single implant (e.g., single leadless microstimulator, including those described herein) may be adapted for sub-diaphragmatic implantation. Such implants may be adapted for sub-diaphragmatic implantation by including one or more features including: a location and/or orientation emitter (configured to emit a wireless signal indicating implant location and orientation, particularly of an inductive coil(s) within the implant), multiple inductive coils for communication (including charging), a controller adapted to deliver a large stimulation dose (e.g., a single pulse or bursts of pulses having between 6.5 and 20 V for a dose duration of between 0.1 second and 1000 seconds (e.g., between 0.3 s and 500 s, between 0.5 s and 100 s, etc.), followed by a low-power, “off” time during which the implant may not apply stimulation; this off time may be between 1 hour and 48 hours (e.g., between 2 hours and 48 hours, between 3 hours and 48 hours, between 4 hours and 36 hours, greater than 2 hours, greater than 3 hours, greater than 4 hours, etc.), or a daily charge delivered at about 0.2 nanocoulombs to 1 kilocoulomb/day.

### Janus Kinase (JAK) Inhibition

As mentioned above, in addition to patient's that failed TNF inhibition, similar and significant results were seen in patients that had previously seen to be intolerant to or had failed to adequately respond to Janus Kinase (JAK) inhibition. In general, stimulating the neuroimmune anti-inflammatory pathway (e.g., the vagus nerve and/or the splenic nerve) with enough energy to depolarize the neurons and cause an action potential activates the neuroimmune pathway. These neuroimmune pathway may utilize both nerves and immune cells, using neurotransmitters to communicate (Tracey (2002)). These reflexes include, but are not limited to, the cholinergic anti-inflammatory pathway and the intestinal cholinergic anti-inflammatory pathway. The cholinergic anti-inflammatory pathway involves the splenic nerve and the spleen (Olofsson et al. (2012)) while the intestinal cholinergic anti-inflammatory pathway is spleen independent (Caravaca et al. (2022)). When neurotransmitters, specifically acetylcholine, is released in the tissues, it binds through the alpha 7 nicotinic acetylcholine receptor on immune cells to downregulate proinflammatory cytokines through modulating the JAK/STAT pathways (de Jonge et al. (2005), “Stimulation of the vagus nerve attenuates macrophage activation by activating the Jak2-STAT3 signaling pathway,” Nat Immunol 6(8): 844-851; and Yang et al. (2015), “Acetylcholine Inhibits LPS-Induced MMP-9 Production and Cell Migration via the alpha7 nAChR-JAK2/STAT3 Pathway in RAW264.7 Cells,” Cell Physiol Biochem 36(5): 2025-2038).

JAK/STAT is an important tyrosine kinase and transcription factor, involved in the production of cytokines central to the pathogenesis and pathophysiology of many inflammatory diseases, including rheumatoid arthritis, psoriatic arthritis, Crohn's disease, and ulcerative colitis. Indeed, several therapies targeting the JAK/STAT pathways have been demonstrated effective and are currently marketed for the treatment of these and other diseases. The JAK/STAT pathway has also been implicated in the neuroimmune anti-inflammatory pathway, and one would think that a patient who has previously not had an adequate clinical response to JAK inhibitors would not be very sensitive to alternative treatments that also modulate JAK/STAT signaling. It is therefore quite surprising that using energy to activate the vagus as described above, my result in a therapeutically beneficial effect in clinical trial subjects that have previously not been adequately treated by JAK/STAT pathway inhibitors.

### EXAMPLES

In the examples below, patients were treated with an implantable electrical stimulator for applying electrical energy to a nerve of the neuroimmune anti-inflammatory pathway (such as the vagus nerve, the sacral nerve, and/or a splenic nerve). Energy was applied on demand by patients from an implanted device including a pulse generator and one or more electrodes, and patients were monitoring for a year or more. In some cases, patients were given one or more drugs (e.g., TNF inhibitor drugs, etc.) in addition to the electrical stimulation. Electrical stimulation was provided on-demand based on patient control of the frequency of stimulation and simulation intensity was no greater than an Upper Comfort Level (UCL), as described herein. In general in these examples, the charge transfer used was between about 0.15 mC-2.25 mC per treatment “dose” as applied by the patient.

In general, the majority of patients that failed to adequately respond to TNF inhibitor drugs prior to receiving achieved a clinically significant response to electrical stimulation, e.g., treatment resulted in a change in the DAS28-CRP that is greater than the minimal clinically important difference (MCID).

### Example 1

For example, in an initial clinical trial, 17 rheumatoid arthritis subjects that had prior exposure to, and exhibited insufficient response or intolerance to, at least 1 TNF inhibitor were implanted with an electrical stimulator and allowed to self-administer a treatment regimen to activate a neuroimmune anti-inflammatory pathway; from this initial study, 9 subjects (69%) achieved ACR20 or greater response, and 8 subjects (62%) had a clinical response that exceeded the DAS28-CRP MID to vagus nerve stimulation.

Subject A3 entered the study as a 59-year-old female with high disease activity. Prior to enrollment this subject was exposed to and had insufficient response or intolerance to adalimumab (TNF inhibitor) and to tocilizumab (TL-6 inhibitor) and to abatacept (CTLA-4). With vagus nerve stimulation alone, this subject did not exceed the DAS28-CRP MID but did achieve ACR20 response after the 6-week primary endpoint and achieved moderate disease activity.

Subject A6 entered the study as a 44-year-old female with high disease activity. Prior to enrollment this subject was exposed to and had insufficient response or intolerance to etanercept and infliximab (TNF inhibitors) and to abatacept (CD20). With vagus nerve stimulation alone, this subject exceeded the DAS28-CRP MID after the 6-week primary endpoint and achieved moderate disease activity and ACR20 response.

Subject A7 entered the study as a 46-year-old female with high disease activity. Prior to enrollment this subject was exposed to and had insufficient response or intolerance to etanercept, adalimumab, and infliximab (TNF inhibitors) and to tocilizumab (TL-6 inhibitor). With vagus nerve stimulation alone, this subject exceeded the DAS28-CRP MID after the 6-week primary endpoint and achieved low disease activity and ACR50 response.

Subject A8 entered the study as a 37-year-old female with high disease activity. Prior to enrollment this subject was exposed to and had insufficient response or intolerance to etanercept, adalimumab, and infliximab (TNF inhibitors) and to abatacept (CTLA-4). With vagus nerve stimulation alone, this subject exceeded the DAS28-CRP MID after the 6-week primary endpoint and achieved low disease activity and ACR50 response.

Subject A10 entered the study as a 61-year-old female with moderate disease activity. Prior to enrollment this subject was exposed to and had insufficient response or intolerance to etanercept, adalimumab, golimumab, and infliximab (TNF inhibitors) and to tocilizumab (TL-6) and to rituximab (CD20). With vagus nerve stimulation alone, this subject exceeded the DAS28-CRP MID after the 6-week primary endpoint and achieved low disease activity and ACR20 response.

Subject A11 entered the study as a 47-year-old female with high disease activity. Prior to enrollment this subject was exposed to and had insufficient response or intolerance to etanercept and adalimumab (TNF inhibitors) and to tocilizumab (TL-6). With vagus nerve stimulation alone, this subject exceeded the DAS28-CRP MID after the 6-week primary endpoint and achieved moderate disease activity and ACR70 response.

Subject A12 entered the study as a 56-year-old female with high disease activity. Prior to enrollment this subject was exposed to and had insufficient response or intolerance to etanercept and adalimumab (TNF inhibitors) and to tocilizumab (TL-6) and to rituximab (CD20) and to abatacept (CTLA-4). With vagus nerve stimulation alone, this subject exceeded the DAS28-CRP MID after the 6-week primary endpoint but had remained in high disease activity and ACR20 response.

Subject A13 entered the study as a 68-year-old female with high disease activity. Prior to enrollment this subject was exposed to and had insufficient response or intolerance to adalimumab (TNF inhibitor). With vagus nerve stimulation alone, this subject exceeded the DAS28-CRP MID after the 6-week primary endpoint and achieved remission and ACR50 response.

Subject A14 entered the study as a 38-year-old male with high disease activity. Prior to enrollment this subject was exposed to and had insufficient response or intolerance to adalimumab (TNF inhibitor). With vagus nerve stimulation alone, this subject exceeded the DAS28-CRP MID after the 6-week primary endpoint and achieved moderate disease activity and ACR50 response.

### Example 2

Although the use of a treatment regimen (e.g., electrical stimulation) to activate a neuroimmune anti-inflammatory pathway in a patient that has failed to adequately respond or is intolerant to a TNF inhibitor therapy may potentiate the TNF inhibitor (e.g., drug) therapy, the use of a TNF inhibitor or other drug was not necessary. For example, Crohn's disease patients that have failed to adequately respond or were intolerant to a TNF inhibitor can respond to nerve stimulation without a biologic or targeted therapy onboard. Of 11 subjects that failed to adequately respond or were intolerant to a TNF inhibitor and which were on vagus nerve stimulation for 16 weeks without a biologic or targeted therapy onboard, 7 of 11 (63%) exceeded the CDAI-100 enhanced MID threshold.

Subject B4 entered the study as a 62-year-old male with moderate disease activity with a CDAI of 292, a SES-CD of 30, and a 5 year history of Crohn's disease. Prior to enrollment this subject was exposed to and had insufficient response or intolerance to infliximab and adalimumab (TNF inhibitors). With vagus nerve stimulation alone, this subject exceeded the enhanced CDAI-100 MID after the 16-week primary endpoint and achieved mild disease activity.

Subject B6 entered the study as a 52-year-old male with moderate disease activity with a CDAI of 270, a SES-CD of 13.7, and a 6 year history of Crohn's disease. Prior to enrollment this subject was exposed to and had insufficient response or intolerance to adalimumab (TNF inhibitor). With vagus nerve stimulation alone, this subject exceeded the enhanced CDAI-100 MID after the 16-week primary endpoint and achieved disease remission.

Subject B7 entered the study as a 25-year-old male with moderate disease activity with a CDAI of 398, a SES-CD of 24.5, and a 6 year history of Crohn's disease. Prior to enrollment this subject was exposed to and had insufficient response or intolerance to infliximab and adalimumab (TNF inhibitors). With vagus nerve stimulation alone, this subject exceeded the enhanced CDAI-100 MID after the 16-week primary endpoint and achieved disease remission.

Subject B8 entered the study as a 31-year-old male with moderate disease activity with a CDAI of 261, a SES-CD of 12, and a 13 year history of Crohn's disease. Prior to enrollment this subject was exposed to and had insufficient response or intolerance to infliximab and adalimumab (TNF inhibitors). With vagus nerve stimulation alone, this subject exceeded the enhanced CDAI-100 MID after the 16-week primary endpoint and achieved disease remission.

Subject B10 entered the study as a 38-year-old female with moderate disease activity with a CDAI of 402, a SES-CD of 13.5, and a 15 year history of Crohn's disease. Prior to enrollment this subject was exposed to and had insufficient response or intolerance to infliximab and adalimumab (TNF inhibitors). With vagus nerve stimulation alone, this subject exceeded the enhanced CDAI-100 MID after the 16-week primary endpoint and remained in moderate disease activity, close to the mild disease cutoff.

Subject B12 entered the study as a 29-year-old female with moderate disease activity with a CDAI of 258, a SES-CD of 16.5, and a 15 year history of Crohn's disease. Prior to enrollment this subject was exposed to and had insufficient response or intolerance to infliximab, adalimumab, etanercept, and golimumab (TNF inhibitors), to ustekinumab (IL12/23 inhibitor), and vedolizumab (α4β7 integrin). With vagus nerve stimulation alone, this subject exceeded the enhanced CDAI-100 MID after the 16-week primary endpoint and achieved disease remission.

Subject B13 entered the study as a 34-year-old male with moderate disease activity with a CDAI of 339, a SES-CD of 16, and a 17 year history of Crohn's disease. Prior to enrollment this subject was exposed to and had insufficient response or intolerance to infliximab and adalimumab (TNF inhibitors). With vagus nerve stimulation alone, this subject exceeded the enhanced CDAI-100 MID after the 16-week primary endpoint and achieved mild disease activity.

### Example 3

Patients that failed to adequately respond to a TNF biologic/targeted drug therapy can respond to the addition of nerve stimulation in conjunction with the previously failed biologic or targeted therapy. For example, a subject (Subject C14) that had previously failed to adequately respond to a biological drug and had vagus nerve stimulation added to the maintained drug therapy as co-therapy for 16 weeks and exceeded the CDAI-100 enhanced MID threshold.

Subject C14 entered the study as a 34-year-old male with moderate disease activity with a CDAI of 294, a SES-CD of 24.5, and a 15 year history of Crohn's disease. Prior to enrollment this subject was exposed to and had insufficient response or intolerance to infliximab and adalimumab (TNF inhibitors) and an insufficient response to vedolizumab (α4β7 integrin). Maintaining the steady course of vedolizumab, vagus nerve stimulation was added as a co-therapy. This subject exceeded the enhanced CDAI-100 MID after the 16-week primary endpoint and achieved mild disease activity (borderline remission).

In a pilot clinical trial, many of the subjects that were exposed to and had insufficient response or intolerance to at least 1 TNF inhibitor (and 1 IL-6 inhibitor), 71% (5 out of 7) subjects achieved ACR20 or greater response.

### Example 4

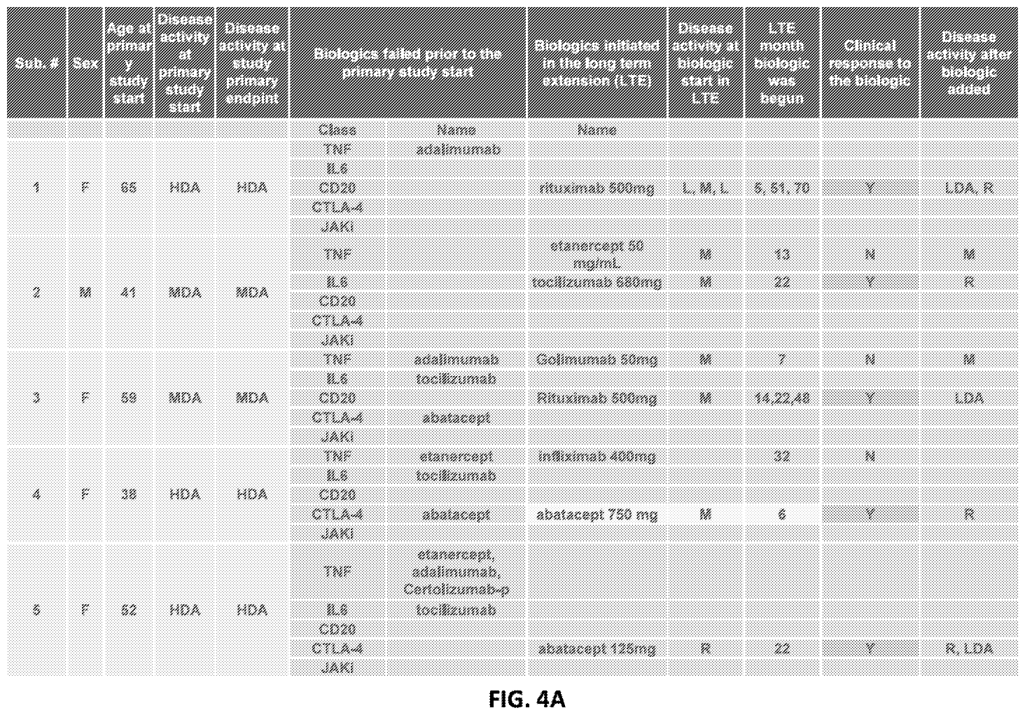

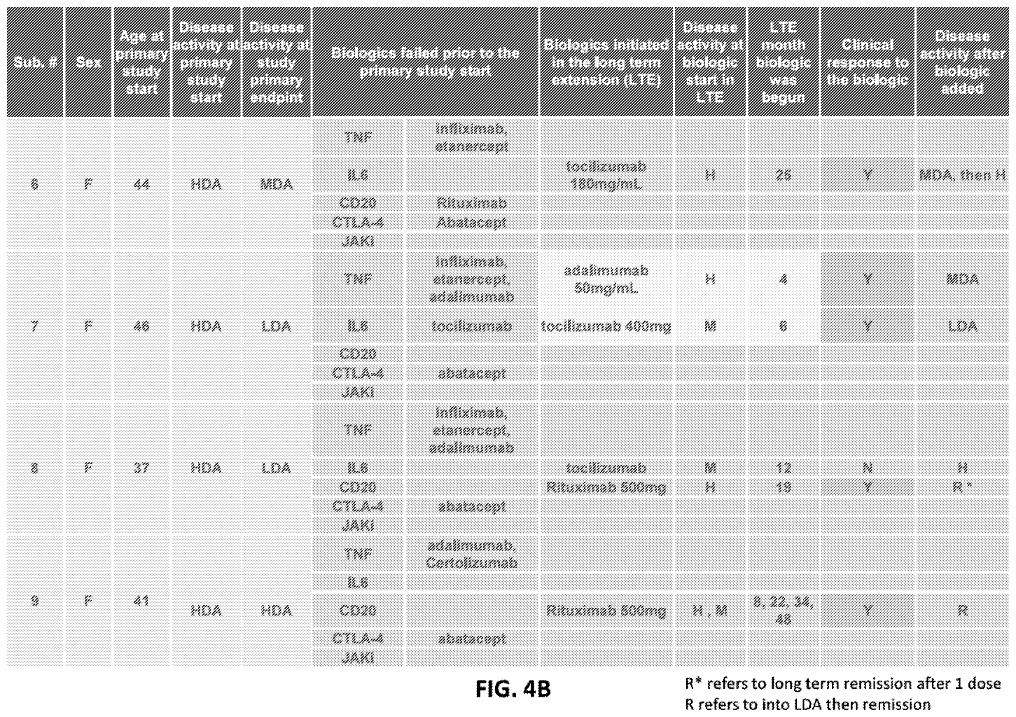

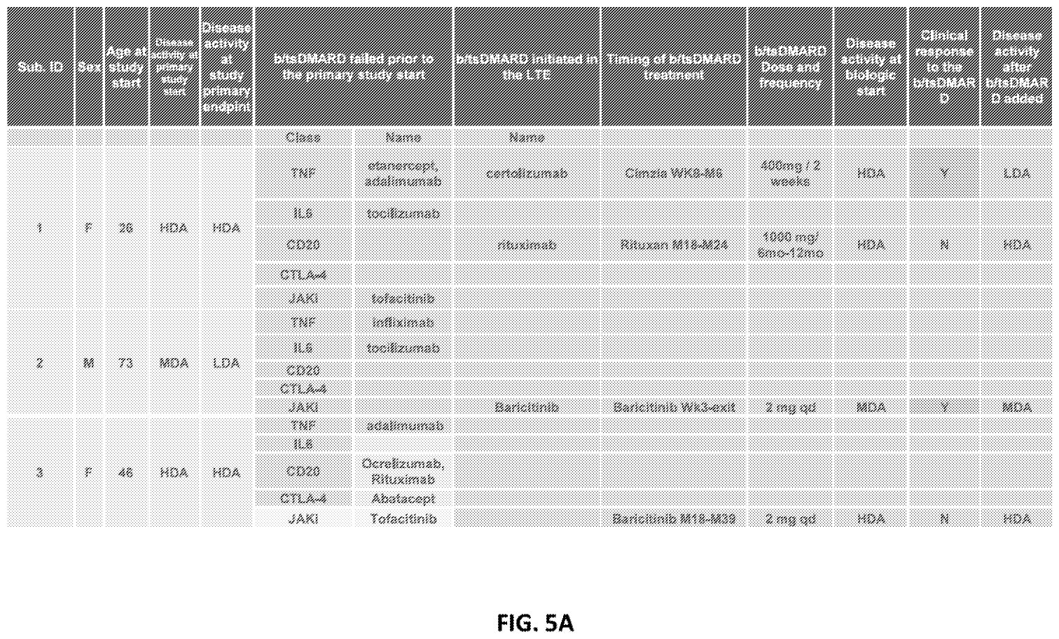

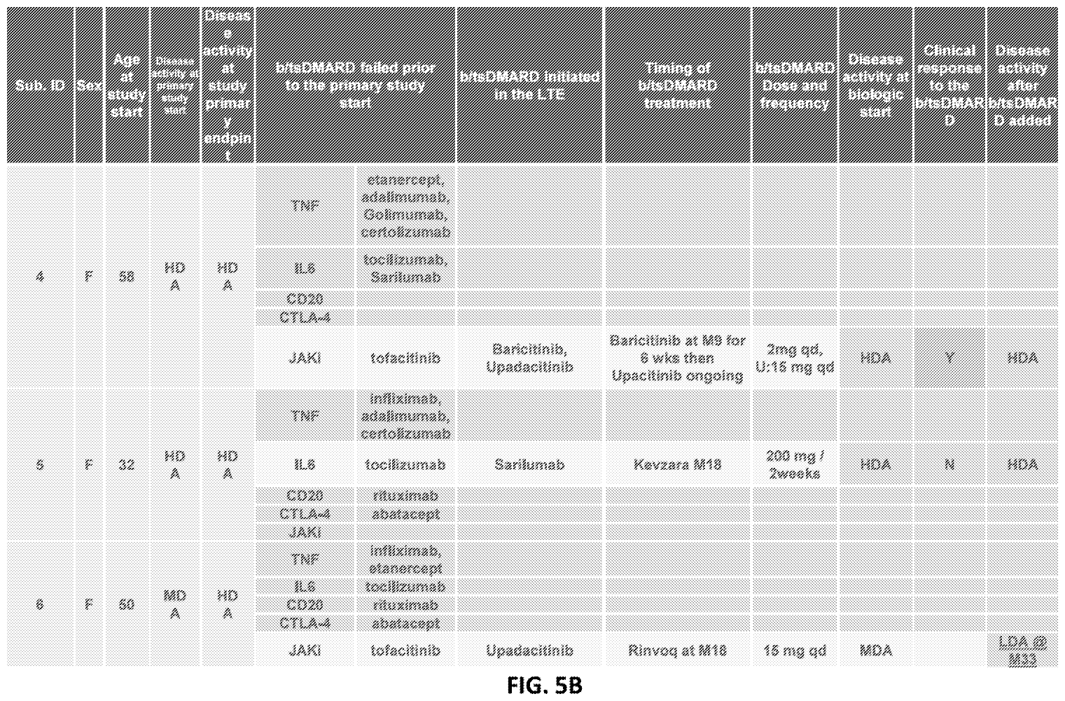

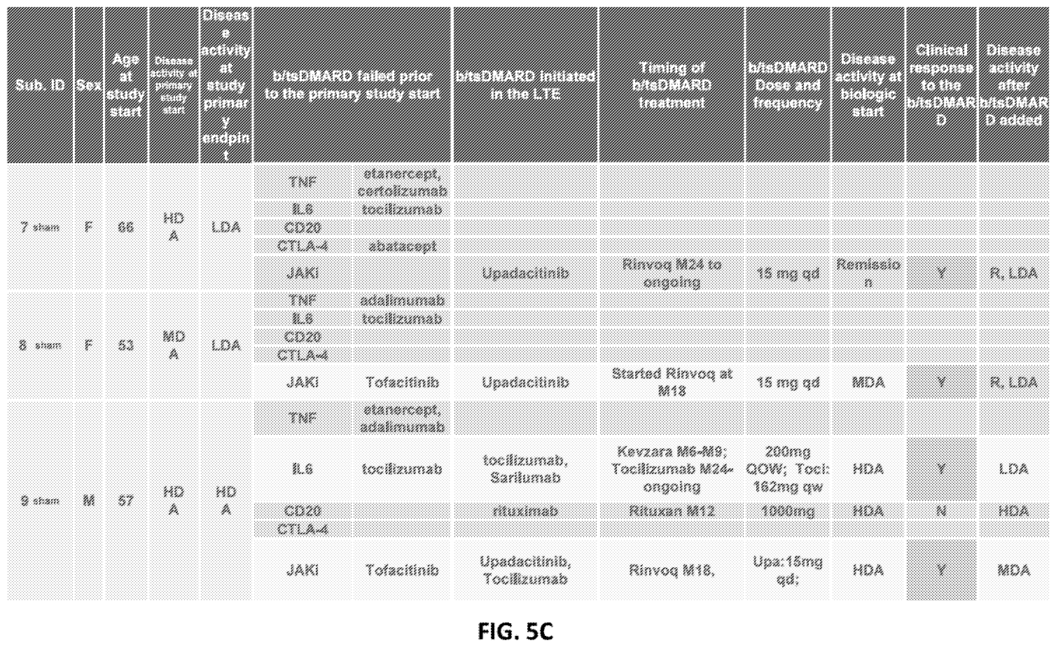

As mentioned above, the methods described herein for treating a patient that had failed or become insensitive to a drug therapy for TNF and/or JAK inhibition surprisingly responded to the combination of a treatment regimen to activate a neuroimmune anti-inflammatory pathway and the same or a similar TNF and/or JAK inhibitor. For example, FIGS. 4A-4B and 5A-5C summarize patient data for patients that were treated as described above. For example, FIGS. 4A-4B show subjects that were treated as describe above, an lists the biologics (shown by class and by name) that were failed prior to implantation of an energy application device to apply energy to activate a neuroimmune anti-inflammatory pathway, as well as biologics that were given in combination with the application of energy during the observed period (e.g., typically greater than 1 year), and an indicator of the response and disease activity (R=remission, LDA=low disease activity, MDA=moderate disease activity, HDA=high disease activity). In FIGS. 5A-5C similar data is provided, showing biologics failed, timing or biologics given in combination with an energy treatment, and patient response (as well as disease activity).

As discussed above, significant responses, e.g., recovery of efficacy of the biologic, for a majority of patients when previously failed biologics or tsDMARDs were provided in combination with the application of energy to activate a neuroimmune anti-inflammatory pathway.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and examples such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or examples of various examples. Combinations of the above examples, and other examples not specifically described herein, will be apparent to those of skill in the art upon reviewing the above description.

1.-61. (canceled)

62. A method of reducing inflammation in a patient that is intolerant to or has failed to adequately respond to either TNF inhibition, the method comprising:
identifying that the patient has failed to respond to TNF inhibitor therapy comprising a drug that inhibits TNF;
administering a treatment regimen to activate a neuroimmune anti-inflammatory pathway in the patient by applying electrical energy to one of: a vagus nerve, a splenic nerve, a radial nerve, a median nerve, an ulnar nerve, a femoral nerve, a sciatic nerve, a tibial nerve, a splanchnic nerve, a phrenic nerve, a hepatic nerve, a renal nerve, a greater auricular nerve, a lesser occipital nerve, or an auriculotemporal nerve, wherein applying electrical energy comprises applying charge ranging from 0.2 nanocoulombs and 5 kilocoulomb per hour.

63. The method of claim 62, wherein applying electrical energy comprises applying electrical energy directly, transdermally applying electrical energy, or percutaneously applying electrical energy.

64. The method of claim 62, wherein applying energy comprises applying energy with energy steering.

65. The method of claim 62, wherein applying electrical energy comprises applying to one or more of: the vagus nerve, the splenic nerve, the femoral nerve or the sacral nerve.

66. The method of claim 62, wherein applying electrical energy comprises applying to one or more of the vagus nerve and the splenic nerve, or a nerve that drive activation of the vagus nerve or splenic nerve.

67. The method of claim 62, wherein applying electrical energy comprises applying to one or more nerve ending within an organ in one or more of: the spleen, the liver, the lymph nodes, the stomach, the small bowel, the large intestine, the pancreas, and the thymus.

68. The method of claim 62, wherein the TNF inhibitor therapy is one or more of: etanercept, adalimumab, certolizumab pegol, infliximab, golimumab, and biosimilars thereof.

69. The method of claim 62, wherein the patient has an immune or inflammatory disorder.

70. The method of claim 62, wherein the immune or inflammatory disorder is one of: rheumatoid arthritis, bursitis, gouty arthritis, polymyalgia rheumatica, Crohn's disease, ulcerative colitis, multiple sclerosis, coeliac disease, systemic lupus erythematosus, aplastic anemia, myocarditis, spondyloarthritis, inflammatory myositis, alopecia, psoriasis, hay fever, atherosclerosis, asthma, chronic inflammation, chronic prostatitis, glomerulonephritis, nephritis, pelvic inflammatory disease, reperfusion injury, transplant rejection, and vasculitis.

71. The method of claim 70, wherein applying electrical energy comprises applying a plurality of electrical stimulations each having a current no greater than an upper comfort level.

72.-73. (canceled)

74. The method of claim 62, wherein applying electrical energy comprises applying nerve stimulations of less than 50 mA.

75. The method of claim 62, wherein applying electrical energy comprises applying nerve stimulations of less than 2 mA.

76. The method of claim 62, wherein applying electrical energy comprises applying nerve stimulations ranging from 20 μA to 200 μA.

77. The method of claim 62, wherein applying electrical energy comprises applying nerve stimulations ranging from 200 μA and 3 mA.

78. The method of claim 62, wherein applying electrical energy comprises applying electrical energy from an implanted device.

79.-80. (canceled)

81. The method of claim 79, wherein the at least one electrode comprises a linear array of electrodes or a nonlinear array of electrodes.

82. The method of claim 78, wherein the implanted device is a leadless device comprising a pulse generator and at least one electrode integrated onto the implanted pulse generator.

83. The method of claim 78, wherein the implanted device is a transcutaneously powered leaded devices with a receiver that receives, stores, and/or transforms energy delivered from outside the body.

84. The method of claim 83, wherein the receiver receives electrical transmission, including nearfield, midfield, and far field transmissions at amplitudes between 0.1 uA-500 mA.

85. The method of claim 62, further comprising administering, concurrent with the application of electrical energy, the same drug, or a drug of the same class that inhibits TNF that the patient had previously failed to respond to.

86. A method of reducing inflammation in a patient that is intolerant to or has failed to respond to either TNF inhibition, the method comprising:
identifying that the patient has failed to respond to TNF inhibitor therapy comprising a drug that inhibits TNF;
administering a treatment regimen to activate a neuroimmune anti-inflammatory pathway in the patient by applying energy to one of: a vagus nerve, a splenic nerve, a radial nerve, a median nerve, an ulnar nerve, a femoral nerve, a sciatic nerve, a tibial nerve, a splanchnic nerve, a phrenic nerve, a hepatic nerve, a renal nerve, a greater auricular nerve, a lesser occipital nerve, or an auriculotemporal nerve; and
administering, concurrent with the application of energy, the same drug, or a drug of the same class that inhibits TNF that the patient had previously failed to respond to.

87.-168. (canceled)
